SEQUENCE LISTING (56 sequences)

SEQ ID NO: 1 - DNA, 135 bp, Artificial Sequence
  Synthetic tracrRNA
aaaaaaagcaccgactcggtgccactttttcaagttgataacggactagccttattttaacttgctatgctgttttgaatggttccaacaagattattttataacttttataacaaataatcaaggagaaattca

SEQ ID NO: 2 - DNA, 4107 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 3 - DNA, 20 bp, Artificial Sequence
  Synthetic crRNA of pCas9-NSI
gcctgaaagcgtgacgagca

SEQ ID NO: 4 - DNA, 799 bp, Synechococcus PCC7942
caatgccttctccaagggcggcattcccctgactgttgaaggcgttgccaatatcaagattgctggggaagaaccgaccatccacaacgcgatcgagcggctgcttggcaaaaaccgtaaggaaatcgagcaaattgccaaggagaccctcgaaggcaacttgcgtggtgttttagccagcctcacgccggagcagatcaacgaggacaaaattgcctttgccaaaagtctgctggaagaggcggaggatgaccttgagcagctgggtcaagtcctcgatacgctgcaagtccagaacatttccgatgaggtcggttatctctcggctagtggacgcaagcagcgggctgatctgcagcgagatgcccgaattgctgaagccgatgcccaggctgcctctgcgatccaaacggccgaaaatgacaagatcacggccctgcgtcggatcgatcgcgatgtagcgatcgcccaagccgaggccgagcgccggattcaggatgcgttgacgcggcgcgaagcggtggtggccgaagctgaagcggacattgctaccgaagtcgctcgtagccaagcagaactccctgtgcagcaggagcggatcaaacaggtgcagcagcaacttcaagccgatgtgatcgccccagctgaggcagcttgtaaacgggcgatcgcggaagcgcggggggccgccgcccgtatcgtcgaagatggaaaagctcaagcggaagggacccaacggctggcggaggcttggcagaccgctggtgctaatgcccgcgacatcttcctgctccagaag

SEQ ID NO: 5 - DNA, 1145 bp, Artificial Sequence
  Synthetic spectinomycin resistance (SpecR) gene
ccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgctggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 6 - DNA, 2663 bp, Artificial Sequence
  Synthetic exogenous gene
aatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatcttaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggctggtgaccacgtcgtggaatgccttcgaattcaggagatataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacatatggcggccgcctgcagctggcgccatcgatacgcgtacgtcgcgaccgcggacatgtacagagctcgagaagtactagtggccacgtgggccgtgcaccttaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgggat

SEQ ID NO: 7 - DNA, 703 bp, Synechococcus PCC7942
aaggcagcgctacccaaatcgctagcttgctggagaagctgaaacaaaccacgggcattgatctggcgaaatccctaccgggtcaatccgactcgcccgctgcgaagtcctaagagatagcgatgtgaccgcgatcgcttgtcaagaatcccagtgatcccgaaccataggaaggcaagctcaatgcttgcctcgtcttgaggactatctagatgtctgtggaacgcacatttattgccatcaagcccgatggcgttcagcggggtttggtcggtacgatcatcggccgctttgagcaaaaaggcttcaaactggtgggcctaaagcagctgaagcccagtcgcgagctggccgaacagcactatgctgtccaccgcgagcgccccttcttcaatggcctcgtcgagttcatcacctctgggccgatcgtggcgatcgtcttggaaggcgaaggcgttgtggcggctgctcgcaagttgatcggcgctaccaatccgctgacggcagaaccgggcaccatccgtggtgattttggtgtcaatattggccgcaacatcatccatggctcggatgcaatcgaaacagcacaacaggaaattgctctctggtttagcccagcagagctaagtgattggacccccacgattcaaccctggctgtacgaataaggtctgcattccttcagagagacattgccatgccg

SEQ ID NO: 8 - DNA, 545 bp, Artificial Sequence
  Synthetic Smt promoter
atccccaagcattcttgggcatgacagagcacgatgctactgcgatcgccccgaccactccccagccgatttctgcctaaggtgcatctctagcgacactcttgtaagtgatcgagggcgttttgataaagcgccacaatgtgatgatcctgtagctggtagtagacatgccgcccttgcttgcgatagctcaccagccgcagattacggagcgatcgcaattggtgagacaccgccgattcggaaacaccaattgcctgggccaaatccccaacacagagctccgatcgcgctaacagggacagcaaccgcagtcgatttggatcggccagcactgcaaaaaattcggctagcgattgggcaacttcgggtgcgatcgcttgaagctccgaggcgatcgccgcatgagtcccttggcagactaccgtctctccgtcctgcagcactggttttgtcatgagccaatcacggtttgtccacccaccatacctgaatcaagattcagatgttaggctaaacacatgaacagttattcagatattcaa

SEQ ID NO: 9 - DNA, 709 bp, Artificial Sequence
  Synthetic LtetO1 promoter
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaagaattcaaa

SEQ ID NO: 10 - DNA, 97 bp, Artificial Sequence
  Synthetic ConII-ribo promoter
accggtttcgaattgacaattaatcatcggctcgtataatggtaccggtgataccagcatcgtcttgatgcccttggcagcaccctgcgaaggaggt

SEQ ID NO: 11 - DNA, 1503 bp, Artificial Sequence
  Synthetic Trc promoter
gacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatcttaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggctggtgaccacgtcgtggaatgcc

SEQ ID NO: 12 - DNA, 1564 bp, Artificial Sequence
  Synthetic LlacO1 promoter
gcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagaattgtgagcggataacaattgacattgtgagcggataacaagatactgagcacatcagcaggacgcactgacc

SEQ ID NO: 13 - DNA, 1190 bp, Artificial Sequence
  Synthetic BAD promoter
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccat

SEQ ID NO: 14 - DNA, 47 bp, Artificial Sequence
  Synthetic Trc′ promoter
accggtttcgaattgacaattaatcatccggctcgtataatggtacc

SEQ ID NO: 15 - DNA, 151 bp, Artificial Sequence
  Synthetic LlacO1′ promoter
ggcgatataggcgccagcaaccgcacctgtggcgccggtgatgccggccacgatgcgtccggcgtagaggatcgagaattgtgagcggataacaattgacattgtgagcggataacaagatactgagcacatcagcaggacgcactgacca

SEQ ID NO: 16 - DNA, 46 bp, Artificial Sequence
  Synthetic ConII promoter
accggtttcgaattgacaattaatcatcggctcgtataatggtacc

SEQ ID NO: 17 - DNA, 37 bp, Artificial Sequence
  Synthetic J23101 promoter
tttacagctagctcagtcctaggtattatgctagcta

SEQ ID NO: 18 - DNA, 35 bp, Artificial Sequence
  Synthetic J23119 promoter
ttgacagctagctcagtcctaggtataatactagt

SEQ ID NO: 19 - DNA, 799 bp, Synechococcus PCC7942; identical to SEQ ID NO: 4

SEQ ID NO: 20 - DNA, 4107 bp, Streptococcus pyogenes
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 21 - DNA, 720 bp, Artificial Sequence
  Synthetic eyfp gene
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 660 bp, Artificial Sequence
  Synthetic chloramphenicol resistance (CmR) gene
ttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccat

SEQ ID NO: 23 - DNA, 783 bp, Synechococcus PCC7942
cggggagtccctgctcgtcacgctttcaggcaccgtgccagatatcgacgtggagtcgatcactgtgattggcgaaggggaaggcagcgctacccaaatcgctagcttgctggagaagctgaaacaaaccacgggcattgatctggcgaaatccctaccgggtcaatccgactcgcccgctgcgaagtcctaagagatagcgatgtgaccgcgatcgcttgtcaagaatcccagtgatcccgaaccataggaaggcaagctcaatgcttgcctcgtcttgaggactatctagatgtctgtggaacgcacatttattgccatcaagcccgatggcgttcagcggggtttggtcggtacgatcatcggccgctttgagcaaaaaggcttcaaactggtgggcctaaagcagctgaagcccagtcgcgagctggccgaacagcactatgctgtccaccgcgagcgccccttcttcaatggcctcgtcgagttcatcacctctgggccgatcgtggcgatcgtcttggaaggcgaaggcgttgtggcggctgctcgcaagttgatcggcgctaccaatccgctgacggcagaaccgggcaccatccgtggtgattttggtgtcaatattggccgcaacatcatccatggctcggatgcaatcgaaacagcacaacaggaaattgctctctggtttagcccagcagagctaagtgattggacccccacgattcaaccctggctgtacgaataaggtctgcattccttcagagagacattgccatgccg

SEQ ID NO: 24 - DNA, 925 bp, Synechococcus PCC7942
cgatcgctttgggacttggaacggtcccttccgggacgatattcggcgtttttggcgtggagatcagggctgtacttacgccctcagtcaacgcctgctgggtagccccgatgtctacagcacagaccaatggtatgccggacgcaccattaacttcatcacctgccatgacggctttacgctgcgagatctagtcagctatagccagaagcacaactttgccaatggagagaacaatcgggacgggaccaatgacaactacagctggaactacggcattgaaggcgagaccgatgaccccacgattctgagcttacgggaacggcagcagcgcaatttgctcgccacgttattcctcgcccagggcacaccgatgctgacgatgggcgatgaggtcaaacgcagtcagcagggtaacaataacgcctactgccaagacaatgagatcagctggtttgattggtcgctgtgcgatcgccatgccgatttcttggtgttcagtcgccgcctgattgaactttcccagtcgctggtgatgttccaacagaacgaactgctgcagaacgaaccccatccgcgtcgtccctatgccatctggcatggcgtcaaactcaaacaacccgattgggcgctgtggtcccacagtctggccgtcagtctctgccatcctcgccagcaggaatggctttacctagcctttaatgcttactgggaagacctgcgcttccagttgccgaggcctcctcgcggccgcgtttggtatcgcttgctcgatacttcactgccgaatcttgaagcttgtcatctgccggatgaggcaaaaccctgcctacggcgcgattacatcgtcccagcgcgatcgctcttactgttgatggctcgtgcttaaaaacaatgcaaacttcaccgtttcagctggtgattt

SEQ ID NO: 25 - DNA, 795 bp, Artificial Sequence
  Synthetic kanamycin resistance (KmR) gene
tcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcat

SEQ ID NO: 26 - DNA, 1045 bp, Synechococcus PCC7942
aaccgttcctgcgcgatcgctcttactgttgatggctcgtgcttaaaaacaatgcaaccctaaccgtttcagctggtgattttcggacgatttggcttacagggataactgagagtcaacagcctctgtccgtcattgcacacccatccatgcactggggacttgactcatgctgaatcacatttcccttgtccattgggcgagaggggaggggaatcttctggactcttcactaagcggcgatcgcaggttcttctacccaagcagtggcgatcgcttgattgcagtcttcaatgctggcctctgcagccatcgccgccaccaaagcatcgtaggcgggacgttgttgctccagtaaagtcttcgcccgtaacaatccccagcgactgcgtaaatccgcttcggcaggattgcgatcgagttgccgccacagttgtttccactgggcgcgatcgtcagctcccccttccacgttgccgtagaccagttgctctgccgctgcaccggccatcaacacctgacaccactgttccagcgatcgctgactgagttgcccctgtgcggcttcggcttctagcgcagctgcttggaactgcacacccccgcgaccaggttgtccttggcgcagcgcttcccacgctgagagggtgtagcccgtcacgggtaaccccagcgcggttgctaccaagtagtgacccgcttcgtgatgcaaaatccgctgacgatattcgggcgatcgctgctgaatgccatcgagcagtaacgtggcaccccgcccctgccaagtcaccgcatccagactgaacagcaccaagaggctaaaacccaatcccgccggtagcagcggagaactacccagcattggtcccaccaaagctaatgccgtcgtggtaaaaatcgcgatcgccgtcagactcaagcccagttcgctcatgcttcctcatctaggtcacagtcttcggcgatcgcatcgatctgatgctgcagcaagcgttttccataccggcgatcgcgccgtcgccctttcgctgccgtggcccgctta

SEQ ID NO: 27 - DNA, 465 bp, Artificial Sequence
  Synthetic sgRNA of psgRNA::O plasmid
ttgacagctagctcagtcctaggtataatactagtgttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgtttta

SEQ ID NO: 28 - DNA, 485 bp, Artificial Sequence
  Synthetic sgRNA of psgRNA::P1 plasmid
ttgacagctagctcagtcctaggtataatactagtattaattgtcaattcgaaacgttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgtttta

SEQ ID NO: 29 - DNA, 485 bp, Artificial Sequence
  Synthetic sgRNA of psgRNA::NT1 plasmid
ttgacagctagctcagtcctaggtataatactagtccgtccagctcgaccaggatgttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgtttta

SEQ ID NO: 30 - DNA, 485 bp, Artificial Sequence
  Synthetic sgRNA of psgRNA::NT2 plasmid
ttgacagctagctcagtcctaggtataatactagtgcgctcctggacgtagccttgttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgtttta

SEQ ID NO: 31 - DNA, 20 bp, Artificial Sequence
  Synthetic crRNA of pCas9O
actcgagcttctggagcagg

SEQ ID NO: 32 - DNA, 20 bp, Artificial Sequence
  Synthetic P1 primer
cggggcaaggcagcttggaa

SEQ ID NO: 33 - DNA, 23 bp, Artificial Sequence
  Synthetic P2 primer
atataccatggtgagcaagggcg

SEQ ID NO: 34 - DNA, 20 bp, Artificial Sequence
  Synthetic P3 primer
tcaacgtaaatgcatgcggg

SEQ ID NO: 35 - DNA, 20 bp, Artificial Sequence
  Synthetic P4 primer
gcatggatctgaccaacatg

SEQ ID NO: 36 - DNA, 410 bp, Synechococcus PCC7942
caatgccttctccaagggcggcattcccctgactgttgaaggcgttgccaatatcaagattgctggggaagaaccgaccatccacaacgcgatcgagcggctgcttggcaaaaaccgtaaggaaatcgagcaaattgccaaggagaccctcgaaggcaacttgcgtggtgttttagccagcctcacgccggagcagatcaacgaggacaaaattgcctttgccaaaagtctgctggaagaggcggaggatgaccttgagcagctgggtcaagtcctcgatacgctgcaagtccagaacatttccgatgaggtcggttatctctcggctagtggacgcaagcagcgggctgatctgcagcgagatgcccgaattgctgaagccgatgcccaggctgcctctgcgatccaaa

SEQ ID NO: 37 - DNA, 400 bp, Synechococcus PCC7942
gcttcaaactggtgggcctaaagcagctgaagcccagtcgcgagctggccgaacagcactatgctgtccaccgcgagcgccccttcttcaatggcctcgtcgagttcatcacctctgggccgatcgtggcgatcgtcttggaaggcgaaggcgttgtggcggctgctcgcaagttgatcggcgctaccaatccgctgacggcagaaccgggcaccatccgtggtgattttggtgtcaatattggccgcaacatcatccatggctcggatgcaatcgaaacagcacaacaggaaattgctctctggtttagcccagcagagctaagtgattggacccccacgattcaaccctggctgtacgaataaggtctgcattccttcagagagacattgccatgccg

SEQ ID NO: 38 - DNA, 97 bp, Synechococcus PCC7942
caatgccttctccaagggcggcattcccctgactgttgaaggcgttgccaatatcaagattgctggggaagaaccgaccatccacaacgcgatcgag

SEQ ID NO: 39 - DNA, 103 bp, Synechococcus PCC7942
ctctctggtttagcccagcagagctaagtgattggacccccacgattcaaccctggctgtacgaataaggtctgcattccttcagagagacattgccatgccg

SEQ ID NO: 40 - DNA, 50 bp, Synechococcus PCC7942
caatgccttctccaagggcggcattcccctgactgttgaaggcgttgcca

SEQ ID NO: 41 - DNA, 53 bp, Synechococcus PCC7942
ccctggctgtacgaataaggtctgcattccttcagagagacattgccatgccg

SEQ ID NO: 42 - DNA, 18 bp, Artificial Sequence
  Synthetic P5 primer
gggcaaggcagcttggaa

SEQ ID NO: 43 - DNA, 22 bp, Artificial Sequence
  Synthetic P6 primer
ccgcatggatctgaccaacatg

SEQ ID NO: 44 - DNA, 20 bp, Artificial Sequence
  Synthetic crRNA of pCas9-glgc
gccctgagttcatccccttt

SEQ ID NO: 45 - DNA, 624 bp, Synechococcus PCC7942
aaaatcagaggcgcgcttttcatcgatcggcaagaccgagagtgtgatgtcggcattggtgtctcggtgccgctgaatgaactggctatagtccatgcggtagagatgatcccccgacaggatcaggtactcatccacatcccactctttgattagccagagatactggcgaaccgcatcggcggtgccttggaaccagttggggttctccggcgtaatctgagctgctagcacctcaacaaagccattgccaaagccgctggagaggttgtaggtctgactgaggtggcggttgagcgaggcagagttaaactgcgtcagcacatagattttgttgatgtcagcgttgatgcaattgctgacgggaatatcgatcaagcggtatttgcccgccagggggaccgctggtttggcgcgctgtttggttagtggatagagacgactgcctgcgcctccaccgagaatgatcgccagcacgtttttcacaatcaatctcccccaagtcaagcggcgctgagacccagtgtctgccggtgagtcagtcttggcaagcaaactgtgcctttgcgatttcttaccctacgcagctccgggatcgatcggaggtaaccaaggctacggaca

SEQ ID NO: 46 - DNA, 708 bp, Synechococcus PCC7942
ttagatcaccgtgttgtcgggaataaccgcccctttgaccacaacgacaatgccgctgcggatcataaagcccagatcttcgcgatcggcctcttccacatggtctttgttgacgatctgaacgttctggccaatgtgggcatttttgtcgacgatggcgcgacggatggtgctgccactgccgattcccatcggcactttgccattggcccgattctggtgccgtagctccgaggtttcgtagaagtcagcgcccatcaacaaggcgtcctggatcacgcagtcggcctcaatgcgggagcgaatccctaagacggaattctgaacggtgcattgcttgagaatgcagccctcgccaatgatcgactgggtcacctggcaatcgagcagcttggttggcggcaggtagcgagcgcgggtgtaaatcggcgcctgctcgtcgtagaagctgaagggtgggctaggttgctgagtcagcgccagattggcctcgtagaatgaggcgatcgtgccgatgtcttcccagtagtcgttgaacagaaaggcttgggtgttgtagcgggtggctgcagcgggaatcacttccttgccaaagtcggtttgctcgggatgatggctgagcaaatcgatcagaacatcccgcttgaagacgtagatgcccatcgaggcaatgaagggctgggcagcagcagcgacagggtcaagccc

SEQ ID NO: 47 - DNA, 1145 bp, Artificial Sequence
  Synthetic gentamycine resistance (GmR) gene
ttatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagataccagcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctgg

SEQ ID NO: 48 - DNA, 101 bp, Artificial Sequence
  Synthetic trc promoter
cacaccacctcaaattgggaatttgtccaagattttcccagtctgccaaagggggtatgaaattcaggtgaaaatctccttcccccgctaagatcagaccg

SEQ ID NO: 49 - DNA, 1161 bp, Synechococcus PCC7942
tcaatcagattcgatcgccaaatcccgatcggcgatcggggtgtagtcgaggttgtggctgcccgtgtagatctgagtggggcggaagatccgattttcgttcagctgctctttccagtgggcgagccagcccgcaacccgcgcgatcgcaaacaccggtgtgaatagatcgctaggaataccgagcttgcgatagaccaagccggagtagaaatcgacgttggggtaaatgcccttgtggctgagtcgctcggctgctgccttttcgactgcgaccgcgatttcgtagtagggatcatggccgaagatatcgaacagttgctcggccagattttgcagaatgactgcccgcggatccttgactttgtagacacggtgcccaaagcccataatgcgcgttttggtggcaatgcagtggtcgaggtagggctcaacattctcgacggaaccgatcgcctccagcatgtccagcacttcttcattggcgccgccatggagggggccagccaaggtgccaacggcagaagcaacgacagcgtagggatcggtcagggtcgaagctgtgaccatcgccgagaaggtcgaggcgttgatcgtatgttcggcgtgcagggtgaggcaaatatcaaaaatccgagctgcgactggatcgggctcgcgctccgtcagcatgtagagaaagttggcggcgtagtccagttcatcgcggggctgaattgggtcgttacccttgcggatcagctggaaggcagccaccatcgtcggaattttggctagcaaacgcacaacggccgcccgaatgtattcgggatcatccaaggcgcgccgcgaatagaacaacccgagggctgcggcgctcgcctgcagggcatccataggatggccgctatcggggaagcatttcatcatgtcccggatgcggaacttgatgcggcggtggtagcgaatttcgtgctcgaactcggtcaattcctgctgagttggtagatggccccaaatcaacaggtaggcggtttccagaaaactgctctgttgcgccagttgctcgatgctgatgccgcgatactctaggacgccgcgctggccatcgacaaagctaatgctcgagagtgtggcgggcacgccttctaggccaggccgaaactcgctgacggcagtcat

SEQ ID NO: 50 - DNA, 3054 bp, Synechococcus PCC7942
tcaacctgtattgcgcatgccagccgcaatgccgttgatcgtcagcaatgctccgcgcagcagttcccctttgctatagcgcgatcgcatcaatcccgtcgtttccgtttgctggcgatactgccgcaggcgtttaagcagggcgacttgcaagaagccgaggggcacgatcgtaccgttgcgcagctgcaccgatcgctgcagttcggggtcgccatccagcaagcgaccgtggttggtaatcgtcaacacgagatgacaagtcagctgaaactctgcagcgatttggctgaacactcgttcaaaccgctcttgatcctcaggattggccagctcatgcacgtagtgatgagcaatctgcaaatcgaccttcgctagggtcatctcgaccttcgagatcaccatgcggaagaacggccacttctcgtagaagtagcgcagcaggttgaggttctgctccgggcgctcctgcaaaaactcctgaagtgctgtgccgacgccataccaagagggcagcagaaaacgactctgcgtccagctaaagacccaggggatggcacgtagactgcccagatcgcgcttgccggttttgcgtcgagccggtcgcgagctgatttgcagtttgctgatctcctcaatcggcgttacctgattgaagaagtcaaccaggtcgggctgctcgtacaccaaagcgcggtaatgtcgccgcgatcgcgccgccaactcttccataatttggttccacggctcaatgtcatcaaagccgctacccagcaggctggactgaatcaccgccgtcgtgatcgtctcaaggttatagagcgccagttcgggcagggcgtatttcgaagccaagacctcgccctgttccgtaatcttgattcggccatctgtggtgcgacccggctgggccaagatcgcctcgtaggcagggccaccacctcgaccgacggagcccccgcggccgtggaagatccgcagcttgacgcggtggtcgcgggctaccgtccctagggctttctgggccttgtggatctcccagttactgctgaggaagcccgagtccttgttactgtcggaataccccagcatcacttcctgcagcggttcagactgggtggggttgagctgggtgtagaagggcagctcaaacagcgccgtcatcacccgcggggcattttgtaagtcctccacagtttcaaacagcggaatcacctgaagcgaactcttgccggtgactgggtcgtagagaccaacctccttggccagcagcagcacttccagcaggtcgctcagctcgcggctcatgctgatgatgtaggtttggcaagccgcctccccaaattcttcctgtagctgcttgaccatccgcagggtttcaatgatctcgcgggtcgattctgagaacggcatgcgcgctggtaccagcggccgacgggttttcagctcttgaaccagccaggcagttcgttctgcttcgctcagctcgttgtagggctggggaagaatttggaggtattcacagatttcgttgatcgcgtcggaatggcgcgagctttcttggcgaatgtcgagatggaccaggttgaaaccaaagatctcgacctggcagatcaacttctctagctcgtaacagctcagtcccgtctgcttaaggttgtgctgaatcagcttgagatcttcgagaaaggccgtaccagcctggtagagggtgccatcccgatattcgggcaggggcgtggtgggatctttttcttgctgctgttggaggcgggtattgcgatcgcgggtgttgtgcaggcgctccagaatataggagagcttgaggcgatagggctcttggcgatagcggaccgctagctgctcataggtctccgggaagagcatgctctcctgttccaacgagctgagcaactccggcagcacctcgctccagtgcatcgagaggctgagcacattgcggagatgttcaaccgctgtgatgtagcgatcgaggacgagactgcgctgatagcaagcggtttgccaagtgacggcagaggtcactgaaggattgccatcgcgatcggagccgacccaagagccgaactggcagaagttgtagcggggcggttgtaggtcggggaaagtcccctgcagcgcgagccgaaagcgctgatagagcagaggaatggcctcaaagaggacttcttggaagtagtggagcgcgtagtccacctcatccaacaccgttggcttgaactggtggagctcatccgtccgccaccagagccgaatctcctcggtcagctgctgacgaatattctgcgcttcaagctctcgaaagcctcggtttttgcctgtttcgagctcatccagttgccgcagcaggtaggcaattcggcgttgtttgtcgcgaatcgtgtggcggacaatttccgtcgggtgagcggtgaaaaccagccgaatatcgaggcgatcggtcagctcttggatcatttgcggcggcacattgagctggcgcaggcgcgggaacagcgtttggaaggttgccggatcttggttgagcggaatcaccgcagcaaagggatcgagctgatgctgaggaatttcaatggtgtgcaatccctccggactttcatctggtcctggcaagggttccaaccgcgatcgctcgtaggcgagttgatattgagtttgctcgtagtgctgctcaacaatattgatcagctgaaagtagagcgcaaaggcacgggcagcccgaatcgcatcgctcaactctagggtttcaatcacctgaaccaaggcttcgccgcccacttctggggcttggccttccggcgaggtcaagtcacgcagctgagtcagaatatcgaccagctcttggccgcactcttggcgcaaaaccacttcccagagttcttcaaccaccgtcaagcgatggcgaaggaaaaactccagcggagacgacagatccaaatccgtggcgaggggactcgaaacggttcggaaagcatcgtcggatgactggagagcagcactcatggcagttcgagcgttctggcagtaattcat

SEQ ID NO: 51 - DNA, 624 bp, Synechococcus PCC7942; identical to SEQ ID NO: 45

SEQ ID NO: 52 - DNA, 708 bp, Synechococcus PCC7942; identical to SEQ ID NO: 46

SEQ ID NO: 53 - DNA, 20 bp, Artificial Sequence
  Synthetic QCas9 F primer
acgcaagtggagtagatgct

SEQ ID NO: 54 - DNA, 20 bp, Artificial Sequence
  Synthetic QCas9 R primer
aggggtcaaacccaatgaca

SEQ ID NO: 55 - DNA, 20 bp, Artificial Sequence
  synthetic qpcr150 f primer
cggccgccatatgttacttg

SEQ ID NO: 56 - DNA, 20 bp, Artificial Sequence
  Synthetic qPCR150 R primer
acaaccactacctgagcacc

(19) United States
(12) Patent Application Publication
(10) Pub. No.: US 2018/0037887 A1
(43) Pub. Date: Feb. 8, 2018

(54) GENE EXPRESSION REGULATION SYSTEM OF SYNECHOCOCCUS ELONGATUS PCC 7942 AND APPLICATION THEREOF

(71) Applicants: NATIONAL TSING HUA UNIVERSITY, Hsinchu (TW); Chang Chun Plastics Co., Ltd., Taipei (TW); Chang Chun Petrochemical Co., LTD., Taipei (TW)

(72) Inventors: Yu-Chen HU, Hsinchu (TW); Hung LI, Hsinchu County (TW); Chun-Hung HUANG, Yunlin County (TW); Li-Yu SUNG, Taoyuan City (TW); Chih-Che SHEN, Hsinchu (TW)

(21) Appl. No.: 15/475,139

(22) Filed: Mar. 31, 2017

(30) Foreign Application Priority Data
Aug. 2, 2016 (TW) 105124446

(51) Int. Cl. C12N 15/113; C12N 15/87; C12N 1/38; C12N 1/20

(52) U.S. Cl. CPC C12N 15/113; C12N 1/20; C12N 15/87; C12N 1/38

(57) ABSTRACT

The present disclosure relates to a gene expression regulation system of a Synechococcus elongatus PCC 7942. The gene expression regulation system of the S. elongatus PCC 7942 includes a S. elongatus PCC 7942 cell, a gene expression interference unit and a gene editing unit. The present disclosure also relates to a method for regulating a gene expression of the S. elongatus PCC 7942. The method includes providing the S. elongatus PCC 7942 cell, using the gene editing unit to insert an exogenous gene into the S. elongates PCC 7942 cell, and using the gene expression interference unit to inhibit an expression of a target gene.

### RELATED APPLICATIONS

This application claims priority to Taiwan Application Serial Number 105124446, filed Aug. 2, 2016, which is herein incorporated by reference.

### SEQUENCE LISTING

The sequence listing submitted via EFS, in compliance with 37 CFR §1.52(e)(5), is incorporated herein by reference. The sequence listing text file submitted via EFS contains the file “CP-3268-US_Sequenceisting”, created on Mar. 29, 2017, which is 57,344 bytes in size.

### BACKGROUND

### Technical Field

The present disclosure relates to a gene expression regulation system of cyanobacteria and an application thereof. More particularly, the present disclosure relates to a gene editing method of the cyanobacteria, a method for interfering gene expression of the cyanobacteria and a method for regulating gene expression of the cyanobacteria.

### Description of Related Art

A lot of biofuels large-scale produced by utilizing microbial genetic engineering are driven by a climate change and an energy crisis. Cyanobacteria live in a wide range of habitats, wherein the cyanobacteria have a variety of properties to adapt to high salt concentration environment, temperature changes dramatically in the environment or high CO₂ concentration environment. Moreover, the cyanobacteria are photoautotrophic prokaryotes capable of directly converting CO₂ into the biofuels via photosynthesis. When compared to heterotrophs, the cyanobacteria do not need to provide additional carbohydrate as a carbon source. They just need sunlight, CO₂, water, nitrogen, phosphorus and trace minerals for their living needs. Therefore, the cyanobacterium is considered one of the most potential microorganisms for producing biofuels in recent years.

The goal of a new generation of genetic engineering has evolved from expressing a single protein by the microorganisms to comprehensively manipulating metabolic pathways of the microorganisms in genetic level for breaking down or producing target product. Therefore, gene knockin and gene knockout at multiple sites on the genome and gene expression regulation are very important issues in the genetic engineering.

The currently widely used gene editing systems include homologous recombination system derived from phage, which is mature and often used in Escherichia coli, and transcription activator-like effector nucleases (TALENs). However, the homologous recombination system derived from phage is limited in length of inserted exogenous gene, wherein DNA fragments larger than 3.5 kb cannot be inserted into the chromosome by the homologous recombination system. In addition, the TALENs is more complex and time-consuming because of a design of enzyme and a change of enzyme. If the plasmid is used as a vector for producing a target protein, an instability of the plasmid and its requirement of antibiotics may affect a stability of gene expression and increase production cost.

At present, the microorganism having capable of high-yield biofuel production can be obtained by metabolic engineering technologies. However, a better way to effectively utilize the microorganisms producing the biofuels is to understand the genes in the chromosomes of the microorganisms, and then inhibit the metabolic pathways which are competed with the target products for optimizing the expressions of the target products, wherein the methods for inhibiting gene expression include the gene knockout and gene knockdown. For inhibiting the expression of the target gene to inhibit other metabolic pathways which are competed with the target product in the cyanobacteria, the homologous recombination system is used to knockout the target gene, and then regulate the metabolic pathway. However, the cyanobacteria possess multiple genome copies per cell, the conventional method cannot clearly and effectively knockout the target gene. In addition, combining with a recombinase system such as FLP/Frt or Cre/Ioxp is required for knocking out multiple target genes (Berla B M, et al. 2013. Synthetic biology of cyanobacteria: unique challenges and opportunities. Front Microbiol 4: 246). Thus an implementation of the conventional method is more complex and takes a longer selection time. Moreover, residual FLP sequence or residual Cre sequence may cause unnecessary gene removal in the next use of the same recombinase system. Therefore, even in the fastest growing cyanobacterium, it takes about three weeks to completely knockout a single target gene. It is also limited by an inability to regulate the target gene and the inability to knockout necessary genes of cells.

### SUMMARY

According to one aspect of the present disclosure, a gene editing system of a Synechococcus elongatus PCC 7942 is provided. The gene editing system of the Synechococcus elongatus PCC 7942 includes a Synechococcus elongatus PCC 7942 cell, a CRISPR/Cas9 expression plasmid and a template plasmid. The CRISPR/Cas9 expression plasmid includes a tracrRNA, a Cas9 gene and a crRNA. The template plasmid successively includes a left homology arm, an antibiotic resistance gene, an exogenous gene and a right homology arm, wherein the left homology arm and the right homology arm compose a homology region, a sequence of the homology region is homologous to a first specific sequence of a chromosome of the Synechococcus elongatus PCC 7942, and a sequence of the crRNA is homologous to a second specific sequence of the chromosome of the Synechococcus elongatus PCC 7942.

According to another aspect of the present disclosure, a gene editing method of a Synechococcus elongatus PCC 7942 includes steps as follows. A CRISPR/Cas9 expression plasmid is constructed. The CRISPR/Cas9 expression plasmid includes a tracrRNA, a Cas9 gene and a crRNA. A template plasmid is constructed. The template plasmid successively includes a left homology arm, an antibiotic resistance gene, an exogenous gene and a right homology arm, wherein the left homology arm and the right homology arm compose a homology region, a sequence of the homology region is homologous to a first specific sequence of a chromosome of the Synechococcus elongatus PCC 7942, and a sequence of the crRNA is homologous to a second specific sequence of the chromosome of the Synechococcus elongatus PCC 7942. The CRISPR/Cas9 expression plasmid and the template plasmid are co-transformed into a Synechococcus elongatus PCC 7942 cell to obtain a transformant. The transformant is cultured, and then the CRISPR/Cas9 expression plasmid therein expresses the tracrRNA, a Cas9 protein and the crRNA to form a Cas9 protein complex, wherein the Cas9 protein complex triggers a double strand break on the second specific sequence of the chromosome of the transformant, and the homology region of the template plasmid and the homology region of the chromosome of the transformant perform a homologous recombination to insert the antibiotic resistance gene and the exogenous gene into the homology region of the chromosome of the transformant.

According to yet another aspect of the present disclosure, a gene expression interference system of a Synechococcus elongatus PCC 7942 is provided. The gene expression interference system of the Synechococcus elongatus PCC 7942 includes a Synechococcus elongatus PCC 7942 cell, a dCas9 expression plasmid and a sgRNA plasmid. The dCas9 expression plasmid successively includes a first left homology arm, a first promoter, a dCas9 gene, a first antibiotic resistance gene and a first right homology arm, wherein the first left homology arm and the first right homology arm compose a first homology region. The sgRNA plasmid successively includes a second left homology arm, a second promoter, a sgRNA, a second antibiotic resistance gene and a second right homology arm, wherein the second left homology arm and the second right homology arm compose a second homology region, a sequence of the sgRNA is homologous to a sequence of a target gene, the target gene is on a chromosome of the Synechococcus elongatus PCC 7942 or on an exogenous plasmid, the second homology region and the first homology region are different, and the second antibiotic resistance gene and the first antibiotic resistance gene are different.

According to still another aspect of the present disclosure, a method for interfering gene expression of a Synechococcus elongatus PCC 7942 includes steps as follows. A dCas9 expression plasmid is constructed. The dCas9 expression plasmid successively includes a first left homology arm, a first promoter, a dCas9 gene, a first antibiotic resistance gene and a first right homology arm, wherein the first left homology arm and the first right homology arm compose a first homology region. A sgRNA plasmid is constructed. The sgRNA plasmid successively includes a second left homology arm, a second promoter, a sgRNA, a second antibiotic resistance gene and a second right homology arm, wherein the second left homology arm and the second right homology arm compose a second homology region, a sequence of the sgRNA homologous to a sequence of a target gene, the target gene is on a chromosome of the Synechococcus elongatus PCC 7942 or on an exogenous plasmid, the second homology region and the first homology region are different, and the second antibiotic resistance gene and the first antibiotic resistance gene are different. The dCas9 expression plasmid is transformed into a Synechococcus elongatus PCC 7942 cell to obtain a first transformant, wherein the first homology region of the dCas9 expression plasmid and the first homology region of the chromosome of the first transformant perform a homologous recombination to insert the first promoter, the dCas9 gene and the first antibiotic resistance gene into the first homology region of the chromosome of the first transformant. The sgRNA plasmid is transformed into the first transformant to obtain a second transformant, wherein the second homology region of the sgRNA plasmid and the second homology region of the chromosome of the second transformant perform the homologous recombination to insert the second promoter, the sgRNA and the second antibiotic resistance gene into the second homology region of the chromosome of the second transformant. The second transformant is cultured and an inducer is added to induce the dCas9 expression plasmid therein to express a dCas9 protein, wherein the dCas9 protein and the sgRNA expressed from the sgRNA plasmid form a dCas9 protein complex, and then the dCas9 protein complex bind to a target gene to inhibit the expression of the target gene.

According to still another aspect of the present disclosure, a gene expression regulation system of a Synechococcus elongatus PCC 7942 is provided. The gene expression regulation system of the Synechococcus elongatus PCC 7942 includes a Synechococcus elongatus PCC 7942 cell, a gene editing unit and a gene expression interference unit. The gene editing unit includes a CRISPR/Cas9 expression plasmid and a template plasmid. The CRISPR/Cas9 expression plasmid includes a tracrRNA, a Cas9 gene and a crRNA. The template plasmid successively includes a first left homology arm, an first antibiotic resistance gene, an exogenous gene and a first right homology arm, wherein the first left homology arm and the first right homology arm compose a first homology region, a sequence of the first homology region is homologous to a first specific sequence of a chromosome of the Synechococcus elongatus PCC 7942, and a sequence of the crRNA is homologous to a second specific sequence of the chromosome of the Synechococcus elongatus PCC 7942. The gene expression interference unit includes a dCas9 expression plasmid and a sgRNA plasmid. The dCas9 expression plasmid successively includes a second left homology arm, a first promoter, a dCas9 gene, a second antibiotic resistance gene and a second right homology arm, wherein the second left homology arm and the second right homology arm compose a second homology region. The sgRNA plasmid successively includes a third left homology arm, a second promoter, a sgRNA, a third antibiotic resistance gene and a third right homology arm, wherein the third left homology arm and the third right homology arm compose a third homology region, a sequence of the sgRNA is homologous to a sequence of a target gene, the target gene is on the chromosome of the Synechococcus elongatus PCC 7942 or on an exogenous plasmid, the third homology region and the second homology region are different, and the third antibiotic resistance gene and the second antibiotic resistance gene are different.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiments, with reference made to the accompanying drawings as follows:

FIG. 1A is a schematic view showing a construction of a CRISPR/Cas9 expression plasmid according to one embodiment of the present disclosure;

FIG. 1B is a schematic view showing how the CRISPR/Cas9 expression plasmid according to one embodiment of the present disclosure operated in a Synechococcus elongatus PCC 7942 cell;

FIG. 2A is analytical results showing how the CRISPR/Cas9 expression plasmid according to one embodiment of the present disclosure affected colony numbers;

FIG. 2B is quantitative analysis results showing how the CRISPR/Cas9 expression plasmid according to one embodiment of the present disclosure affected a death rate;

FIG. 3 is a flow diagram showing a gene editing method of the S. elongatus PCC 7942 according to another embodiment of the present disclosure;

FIG. 4 is a schematic view showing a construction and a transformation of a gene editing system of the S. elongatus PCC 7942 according to one embodiment of the present disclosure;

FIG. 5A shows photographs of antibiotic-resistant colonies after homologous recombining by the gene editing system of the S. elongatus PCC 7942 according to one embodiment of the present disclosure;

FIG. 5B is a bar chart showing numbers of chromosomes of the S. elongatus PCC 7942 which are integrated an exogenous gene by the gene editing system of the S. elongatus PCC 7942 according to one embodiment of the present disclosure;

FIG. 6A shows analytical results of a colony PCR for confirming precise integration of the exogenous gene in the chromosome of the S. elongatus PCC 7942;

FIG. 6B shows analytical results of a qPCR assay for confirming the residual CRISPR/Cas9 expression plasmid of the present disclosure after the transformation;

FIG. 7 shows analytical results of homologous recombination efficiency at different dose combinations of the CRISPR/Cas9 expression plasmid and the template plasmid of the gene editing system of the S. elongatus PCC 7942 co-transformed into the S. elongatus PCC 7942 cell according to one embodiment of the present disclosure;

FIG. 8A is a schematic view showing a construction of the template plasmids with different homology arm lengths;

FIG. 8B is an analytical result showing the homologous recombination efficiency of the gene editing system of the S. elongatus PCC 7942 with different homology arm lengths according to one embodiment of the present disclosure;

FIG. 9A is an analytical result showing average copy number per cell of the chromosome of the S. elongatus PCC 7942 which is integrated the exogenous gene;

FIG. 9B shows analytical results of a colony PCR for confirming an integration of the exogenous gene in all of the chromosome of the S. elongatus PCC 7942 by the gene editing system of the S. elongatus PCC 7942 according to one embodiment of the present disclosure;

FIG. 10 is an analytical result showing a stability of the integration of the exogenous gene in the chromosome of the S. elongatus PCC 7942;

FIG. 11A is a schematic view showing metabolic pathways and regulatory genes of the S. elongatus PCC 7942;

FIG. 11B is a schematic view showing a construction of a plasmid for knocking out a glgc gene of the chromosome of the S. elongatus PCC 7942 by the gene editing system;

FIG. 11C is an analytical result showing a change of glycogen accumulation in the S. elongatus PCC 7942 knocked out the glgc gene;

FIG. 11D is an analytical result showing a change of succinate production in the S. elongatus PCC 7942 knocked out the glgc gene;

FIG. 12A is a schematic view showing constructions of inducible promoters of the gene expression interference system of the S. elongatus PCC 7942 according to yet another embodiment of the present disclosure;

FIG. 12B shows analytical results of the inducible promoters of the gene expression interference system of the S. elongatus PCC 7942 according to yet another embodiment of the present disclosure;

FIG. 13A is a schematic view showing constructions of constitutive promoters of the gene expression interference system of the S. elongatus PCC 7942 according to yet another embodiment of the present disclosure;

FIG. 13B shows analytical results of the constitutive promoters of the gene expression interference system of the S. elongatus PCC 7942 according to yet another embodiment of the present disclosure;

FIG. 14 is a flow diagram showing a method for interfering gene expression of the S. elongatus PCC 7942 according to still another embodiment of the present disclosure;

FIG. 15A is a schematic view showing a construction and a homologous recombination of a dCas9 plasmid according to still another embodiment of the present disclosure;

FIG. 15B is a schematic view showing a construction and homologous recombination of a sgRNA plasmid according to still another embodiment of the present disclosure;

FIGS. 16A and 16B are analytical results showing an expression of a target gene inhibited by the gene expression interference system of the S. elongatus PCC 7942 according to yet another embodiment of the present disclosure;

FIG. 17A is analytical result of a cytotoxicity against the S. elongatus PCC 7942 cells affected by the gene expression interference system of the S. elongatus PCC 7942 according to yet another embodiment of the present disclosure;

FIG. 17B is analytical result of a gene regulation stability of the gene expression interference system of the S. elongatus PCC 7942 according to yet another embodiment of the present disclosure; and

FIG. 18 is a flow diagram showing a method for regulating a gene expression of the S. elongatus PCC 7942 according to yet another embodiment of the present disclosure.

### DETAILED DESCRIPTION

The term “Synechococcus elongatus PCC 7942” refers to a cyanobacterium deposited to the Pasteur Culture Collection under accession number PCC 7942, which is a Gram-negative bacteria. The S. elongatus PCC 7942 is a polyploid monoplast with two endoplasmic plasmids, pANL and pANS, and ring chromosomes, wherein the genome size is about 2.8 Mb, and average copy number of the chromosome is four. Moreover, the S. elongatus PCC 7942 is an obligate autotroph with a long rod shape; it can live in fresh water under low nutrient. An ideal growth temperature for the S. elongatus PCC 7942 is 30° C. Under an appropriate growth environment, the S. elongatus PCC 7942 can replicate once every 24 hours. The S. elongatus PCC 7942 is found to be able to homologously recombine the exogenous DNA into the chromosome by a natural transformation. The S. elongatus PCC 7942 has high transformation efficiency as well as homologous recombination efficiency, thus the S. elongatus PCC 7942 is one strain of the cyanobacteria that can successfully transform the exogenous DNA. Integrated sites of the chromosome of the S. elongatus PCC 7942 often used are neutral site I (NSI) and neutral site II (NSII). Another stains of the cyanobacteria commonly used to produce biofuels are Synechocystis sp. PCC 6803), Synechococcus sp. PCC7002 and Anabaena variabilis PCC 7120. Both the S. elongatus PCC 7942 and the Synechocystis sp. PCC 6803, another strain commonly used in genetic engineering, are the cyanobacteria, but many of their characteristics are different. In the morphology of monomeric cell, the S. elongata PCC 7942 cells often connect by a number of cells to form chains, whereas the cells of the Synechocystis sp. PCC 6803 often aggregate together. A phylogenetic tree analyzed by ribosomal 16S RDNA sequence also shows the difference between the S. elongata PCC 7942 and the Synechocystis sp. PCC 6803. In growth characteristics and chromosomal properties, the S. elongata PCC 7942 is an autotroph with a genome of approximately 2.8 Mb and 4 copies of the chromosome. A cell doubling time of the S. elongata PCC 7942 is 12-24 hours. The Synechocystis sp. PCC 6803 is a facultative autotroph with the genome of approximately 3.6 Mb and a maximum 218 copies of the chromosome. The cell doubling time of the Synechocystis sp. PCC 6803 is 6-12 hours. In addition, the S. elongata PCC 7942 and the Synechocystis sp. PCC 6803 have different preference on the choice of genetic engineering tools (such as promoters). Although the Synechocystis sp. PCC 6803 is earlier understood and used to produce biochemicals, selections of strategies and tools still need to modify based on the characteristics of the S. elongata PCC 7942 in details of practical application.

The term “CRISPR/Cas9” refers to clustered regularly interspaced short palindromic repeats (CRISPRs) and CRISPR-associated protein (Cas9) system. It is an adaptive immunity system derived from prokaryotes to inhibit activities of foreign nucleic acid fragments in the cell and eliminate foreign plasmids or phages. The CRISPR system can be divided into three types according to the mechanisms. The CRISPR/Cas9 system belongs to type II CRISPR system, which is derived from Streptococcus pyogenes. The mechanism of the CRISPR/Cas9 system can be divided into two stages. The first stage is to obtain immunity. The CRISPR/Cas9 system cleaves the foreign nucleic acid fragments invaded cell through virus infection or conjugation, and then integrates the cleavages into CRISPR gene site, which is also known as the “spacer”. The second stage is to inhibit the activity of the foreign nucleic acid fragments. The CRISPR gene site contains multiple spacers that are complementary to the sequence of the target nucleic acid, wherein each spacer encodes a CRISPR RNA (crRNA) and is flanked by a repeat sequence (direct repeats). First, the CRISPR gene transcribes pre-crRNA and binds to trans-activating crRNAs (tracrRNAs). Next, the pre-crRNA-tracrRNA complex is treated with RNase III to become a mature crRNA. The Cas9 protein then chelates with the tracrRNA and the mature crRNA to form a ribonucleoprotein copolymer, and directs the copolymer to the sequence of the target gene that is complementary to the spacer (protospacer) through the spacer on the crRNA. Finally, a blunt-ended double strand break (DSB) is generated at 3 bp upstream of the 3′end of the protospacer by an HNH nuclease domain and a RuvC nuclease domain on the Cas9 protein. For causing the double strand break, the protospacer not only includes the sequence complementary to spacer but also includes a specific protospacer-adjacent motif (PAM) at downstream of its 3′end, wherein the sequence of the PAM is NGG (N represents a random DNA codon) in Streptococcus pyogenes type II CRISPR/Cas9 system.

The term “CRISPRi” refers to CRISPR interference system, which is a modified type II CRISPR/Cas9 system derived from the Streptococcus pyogenes. The Cas9 protein is modified to lose its endonuclease activity (RuvC1 and HNH), known as dCas9 (Cas9 D10A and H841A). The action principle of the CRISPRi system is the same as the type II CRISPR/Cas9 system, wherein the dCas9 protein binds to the target sequence of the target gene by an induction of the sgRNA or crRNA-trancrRNA complex, but the dCas9 protein does not cleave the target gene. Therefore, it can be used to block the RNA polymerase performing a gene transcription and inhibit an expression of the target gene.

Reference will now be made in detail to the present embodiments of the present disclosure, examples of which are illustrated in the accompanying drawings.

### EXAMPLES

### I. A Gene Editing System of the S. elongatus PCC 7942 of the Present Disclosure

### 1.1 Establishment of a CRISPR/Cas9 Gene Editing System of the S. elongatus PCC 7942

The gene editing system of the S. elongatus PCC 7942 of the present disclosure includes a S. elongatus PCC 7942 cell (GeneArt® Synechococcus Engineering Kits; Life technology), a CRISPR/Cas9 expression plasmid and a template plasmid.

The CRISPR/Cas9 expression plasmid includes a tracrRNA, a Cas9 gene and a crRNA. According to one example of this embodiment, the CRISPR/Cas9 expression plasmid is a pCas9-NSI plasmid, wherein the nucleotide sequence of the tracrRNA is referenced as SEQ ID NO: 1, the nucleotide sequence of the Cas9 gene is referenced as SEQ ID NO: 2, and the nucleotide sequence of the crRNA is referenced as SEQ ID NO: 3. The tracrRNA, the Cas9 gene and the crRNA are constructed on pCas9 vector (addgene) to obtain the pCas9-NSI plasmid.

The template plasmid successively includes a left homology arm, an antibiotic resistance gene, an exogenous gene and a right homology arm, wherein the left homology arm and the right homology arm compose a homology region, a sequence of the homology region is homologous to a first specific sequence of a chromosome of the S. elongatus PCC 7942. According to one example of this embodiment, the template plasmid is a pHR-trcS plasmid, wherein the nucleotide sequence of the left homology arm is referenced as SEQ ID NO: 4, the antibiotic resistance gene is a spectinomycin resistance (Spec^{R}) gene having the nucleotide sequence referenced as SEQ ID NO: 5, the nucleotide sequence of the exogenous gene is referenced as SEQ ID NO: 6, and the nucleotide sequence of the right homology arm is referenced as SEQ ID NO: 7. The homology region composed of the left homology arm and the right homology arm is partial sequence of the neutral site I (NSI) gene of the S. elongatus PCC 7942 cell. The left homology arm, the antibiotic resistance gene, the exogenous gene and the right homology arm are constructed on pSYN_1 vector (Life technology) to obtain the pHR-trcS plasmid.

To establish the CRISPR/Cas9 gene editing system of the S. elongatus PCC 7942, it is necessary to test whether the CRISPR/Cas9 expression plasmid can successfully trigger the double strand break of the S. elongatus PCC 7942 cell. The S. elongatus PCC 7942 possesses four copies of the chromosomes, and a cell death of the S. elongatus PCC 7942 can cause by the double strand breaks on all genome copies. Therefore, a death rate can be an indicator of a CRISPR/Cas9 system mediated double strand break.

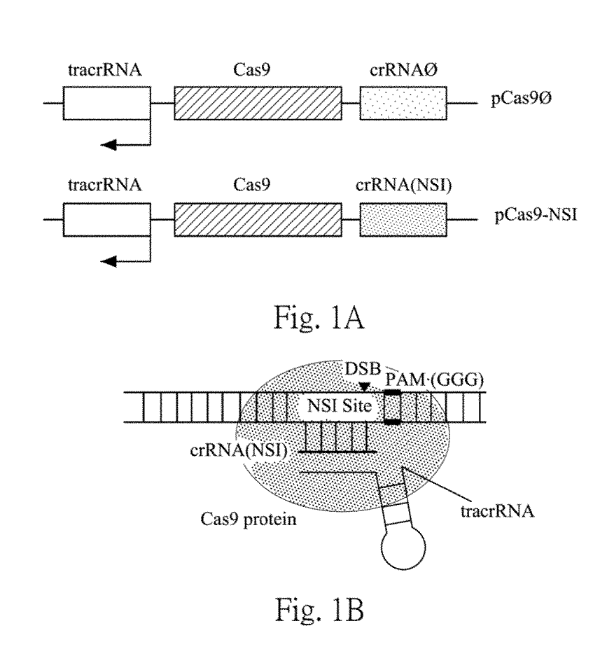

FIG. 1A is a schematic view showing a construction of the CRISPR/Cas9 expression plasmid according to one embodiment of the present disclosure. FIG. 1B is a schematic view showing how the CRISPR/Cas9 expression plasmid according to one embodiment of the present disclosure operated in the S. elongatus PCC 7942 cell. There are two CRISPR/Cas9 expression plasmid, pCas9Ø plasmid and pCas-NSI plasmid, constructed in this example. The nucleotide sequence of the crRNA of the pCas9Ø plasmid is referenced as SEQ ID NO: 31, which does not target any sequence on the chromosome of the S. elongatus PCC 7942 cell. The sequence of the crRNA of the pCas9-NSI plasmid is homologous to partial sequence of the NSI gene of the chromosome of the S. elongatus PCC 7942, which is not on the homology region of the template plasmid. Therefore, the crRNA of the pCas9-NSI can bind to the NSI gene of the chromosome of the S. elongatus PCC 7942 cell and then trigger the double strand break.

To confirm that the CRISPR/Cas9 expression plasmid is indeed effective in the S. elongatus PCC 7942, the constructed plasmid is used for a cutting efficiency analysis based on the death of the S. elongatus PCC 7942 in this example. In the cutting efficiency analysis, the constructed CRISPR/Cas9 plasmid is transformed into the S. elongatus PCC 7942 cell, and then observed whether the Cas9 protein complex triggers the double strand break at a target site of the chromosomes of the S. elongatus PCC 7942. If the double strand break is successfully triggered, the S. elongatus PCC 7942 will die. Thus the colony number of surviving colonies can verify whether the CRISPR/Cas9 expression plasmid works and the CRISPR/Cas9 expression plasmid per se has the cytotoxicity against the S. elongatus PCC 7942 cell.

As tests, the pCas9-NSI plasmid is transformed into the S. elongatus PCC 7942 cell at 250 ng, 500 ng, 1000 ng and 2000 ng, respectively. As controls, the S. elongatus PCC 7942 cell is not transformed any plasmid (control group) or transformed with 1000 ng of the pCas9Ø plasmid, wherein the pCas9Ø plasmid would not trigger the double strand break on the chromosome of the S. elongatus PCC 7942 and can be the control for confirming whether the CRISPR/Cas9 expression plasmids have cytotoxicity against the S. elongatus PCC 7942 cell. An efficiency of the CRISPR/Cas9 system mediated double strand break in the S. elongatus PCC 7942 is verified by the death rate of the S. elongatus PCC 7942.

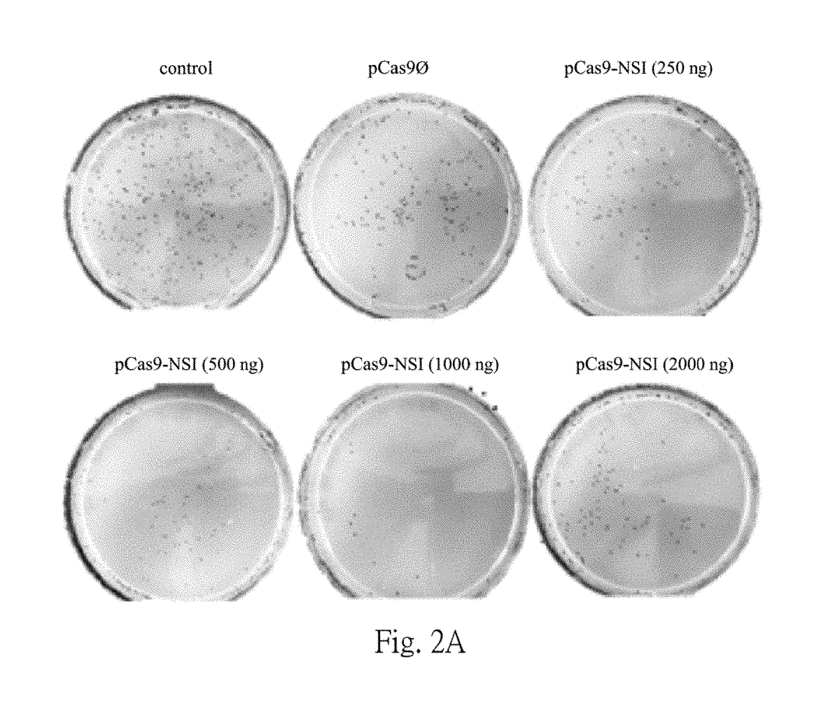

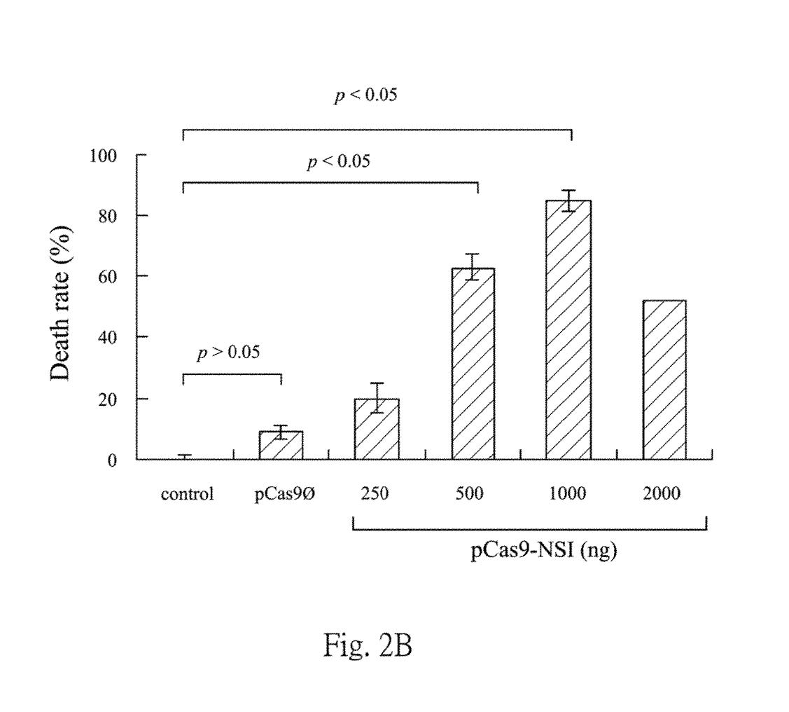

Table 1 shows colony numbers after operating the CRISPR/Cas9 expression plasmid in the S. elongatus PCC 7942 cell, wherein a calculated unit of the colony numbers is a colony forming unit (CFU). FIG. 2A is analytical result showing how the CRISPR/Cas9 expression plasmid affected colony numbers. FIG. 28 is quantitative analysis result showing how the CRISPR/Cas9 expression plasmid affected the death rate, wherein the death rate is calculated by the following formula 1:

$$Death rate ( \% ) = [ 1 - \frac{{CFU}_{test}}{{CFU}_{control}} ] \times 100. \qquad formula I$$

TABLE 1

| Group | Colony numbers (CFU) |
|---|---|
| Control | 3.5 × 10⁸ |
| pCas9Ø plasmid | 3.2 × 10⁸ |
| pCas9-NSI plasmid (250 ng) | 2.8 × 10⁸ |
| pCas9-NSI plasmid (500 ng) | 1.4 × 10⁸ |
| pCas9-NSI plasmid (1000 ng) | 4.2 × 10⁷ |
| pCas9-NSI plasmid (2000 ng) | 2.1 × 10⁸ |

As shown in table 1, FIG. 2A and FIG. 2B, increasing the pCas9-NSI dose gives rise to lower colony number of the S. elongatus PCC 7942 when compared to the control group, and the colony number is the lowest at 1000 ng. The death rate (a double strand break rate) also is culminated at 85.0±3% at 1000 ng. Although the death rate of the S. elongatus PCC 7942 is significantly increased in test group when compared with the control group, the group transformed with the pCas9Ø plasmid results in comparable colony number when compared with the control group. It indicates that the CRISPR/Cas9 expression plasmid does not cause any cytotoxicity against the S. elongatus PCC 7942, and the double strand break of a targeted site is only triggered by adding the crRNA/tracrRNA targeted on the targeted site.

### 1.2 A Gene Editing Method of the S. elongatus PCC 7942

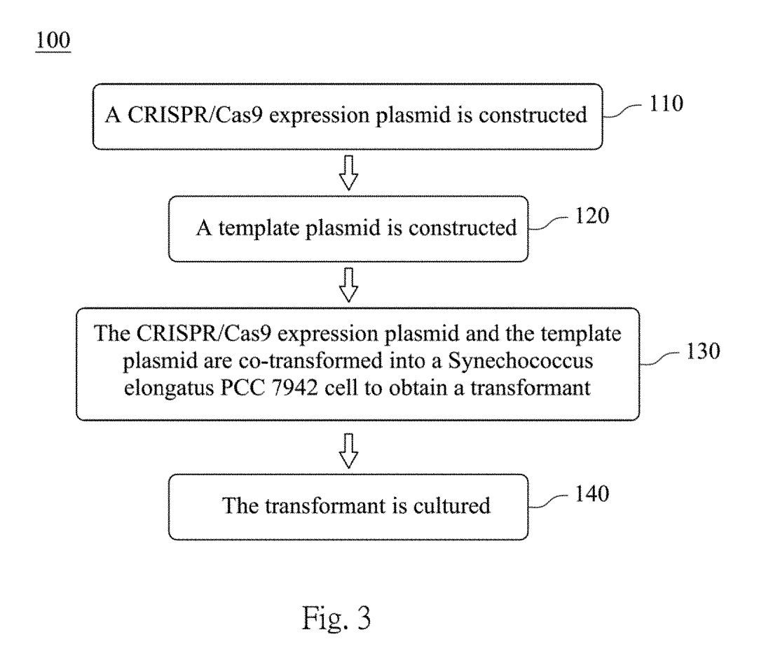

FIG. 3 is the flow diagram showing the gene editing method 100 of the S. elongatus PCC 7942 according to another embodiment of the present disclosure. In FIG. 3, the gene editing method 100 of the S. elongatus PCC 7942 includes a step 110, a step 120, a step 130 and a step 140.

In the step 110, the CRISPR/Cas9 expression plasmid is constructed. The CRISPR/Cas9 expression plasmid includes the tracrRNA, the Cas9 gene and the crRNA.

In the step 120, the template plasmid is constructed. The template plasmid successively includes the left homology arm, the antibiotic resistance gene, the exogenous gene and the right homology arm, wherein the left homology arm and the right homology arm compose a homology region, a sequence of the homology region is homologous to the first specific sequence of a chromosome of the S. elongatus PCC 7942.

In the step 130, the CRISPR/Cas9 expression plasmid and the template plasmid are co-transformed into the S. elongatus PCC 7942 cell to obtain a transformant.

In the step 140, the transformant is cultured, and then the CRISPR/Cas9 expression plasmid therein expresses the tracrRNA, a Cas9 protein and the crRNA to form a Cas9 protein complex, wherein the Cas9 protein complex triggers the double strand break on the second specific sequence of the chromosome of the transformant, and the homology region of the template plasmid and the homology region of the chromosome of the transformant perform a homologous recombination to insert the antibiotic resistance gene and the exogenous gene into the homology region of the chromosome of the transformant.

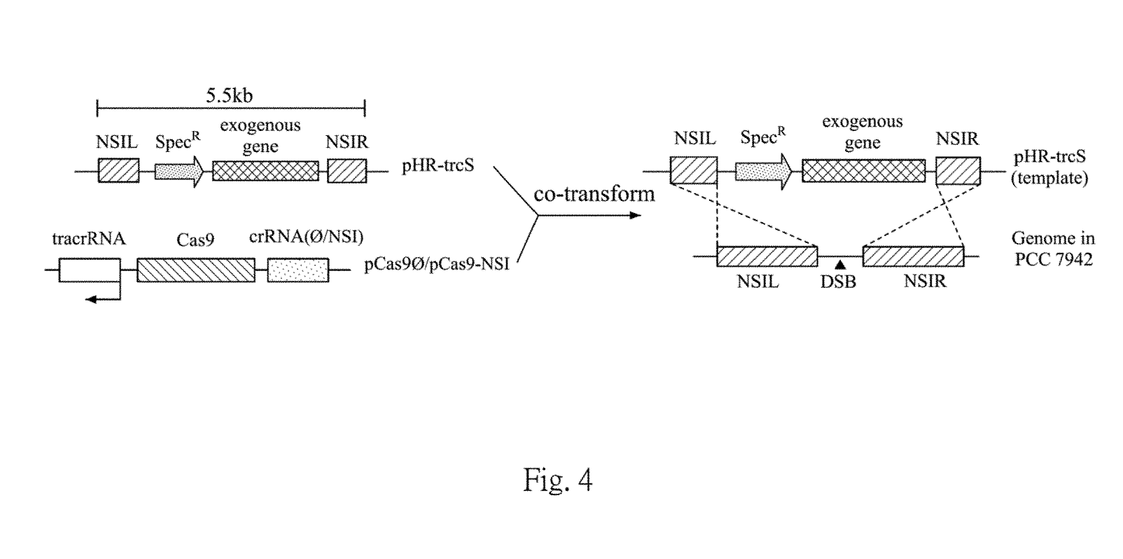

FIG. 4 is a schematic view showing a construction and a transformation of the gene editing system of the S. elongatus PCC 7942 according to one embodiment of the present disclosure. According to one embodiment of this example, the CRISPR/Cas9 expression plasmid is the Cas9-NSI plasmid, and the template plasmid is the pHR-trcS plasmid. The Cas9-NSI plasmid and the pHR-trcS plasmid are co-transformed into the S. elongatus PCC 7942 cell. The Cas9-NSI plasmid triggers the double strand break on the NSI gene of the chromosome of the S. elongatus PCC 7942, and the left homology arm (NSIL) and the right homology arm (NSIR) of the pHR-trcS plasmid and the NSI gene of the chromosome of the S. elongatus PCC 7942 perform the homologous recombination to insert the Spec gene and the exogenous gene into the NSI gene of the chromosome of the S. elongatus PCC 7942.

### 1.3 the CRISPR/Cas9 Gene Editing System Promotes a Homologous Recombination in S. elongatus PCC 7942

To verify that the gene editing system of the S. elongatus PCC 7942 of the present disclosure can enhance a success rate of conventional homologous recombination techniques, a homologous recombination efficiency analysis is performed in this example. The CRISPR/Cas9 expression plasmid and the pHR-trcS plasmid are co-transformed into the S. elongatus PCC 7942 cell to obtain the transformant, and the colony number of the transformant which is inserted the exogenous gene into its chromosomes is observed to verify the homologous recombination efficiency. The pHR-trcS plasmid (2000 ng) and the pCas9-NSI plasmid (500 ng) are co-transformed into the S. elongatus PCC 7942 cell (Cas9-NSI group). As controls, the S. elongatus PCC 7942 cell is transformed with 2000 ng of the pHR-trcS plasmid (HR-trcS group) as a homologous recombination template or co-transformed with 2000 ng of the pHR-trcS plasmid and 500 ng of the pCas9Ø plasmid (Cas9Ø group). The transformants are selected by the medium containing the spectinomycin, and the surviving colonies indicate the number of the transformants integrated the exogenous gene into their chromosome by the homologous recombination.

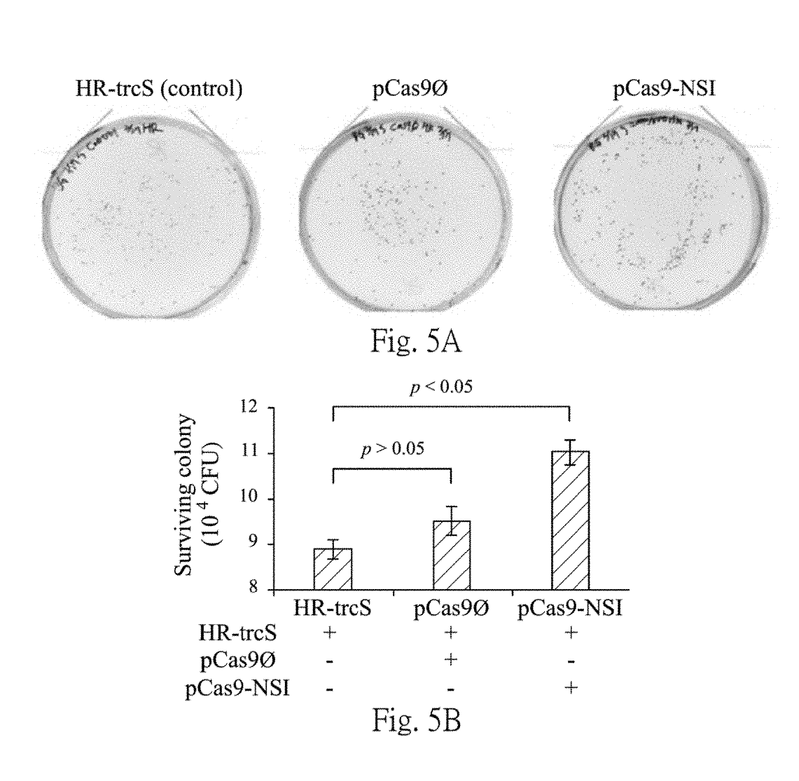

Table 2 shows the colony number after operating the gene editing system of the S. elongatus PCC 7942 of the present disclosure in the S. elongatus PCC 7942 cell. FIG. 5A shows photographs of antibiotic-resistant colonies after homologous recombining by the gene editing system of the S. elongatus PCC 7942 according to one embodiment of the present disclosure. FIG. 5B is a bar chart showing numbers of chromosomes of the S. elongatus PCC 7942 which are integrated the exogenous gene by the gene editing system of the S. elongatus PCC 7942 according to one embodiment of the present disclosure.

TABLE 2

| Group | Colony number (CFU) |
|---|---|
| pHR-trcS plasmid | 8.9 × 10⁴ |
| pHR-trcS plasmid + pCas9Ø plasmid | 9.5 × 10⁴ |
| pHR-trcS plasmid + pCas9-NSI plasmid | 1.1 × 10⁵ |

In Table 2 and FIGS. 5A-5B, the colony number of the surviving colonies of the pCas9-NSI group is higher than that of the HR-trcS group or the Cas9Ø group. It indicates that the gene editing system of the S. elongatus PCC 7942 of the present disclosure indeed can enhance the homologous recombination efficiency to insert the exogenous gene into the chromosome of the S. elongatus PCC 7942.

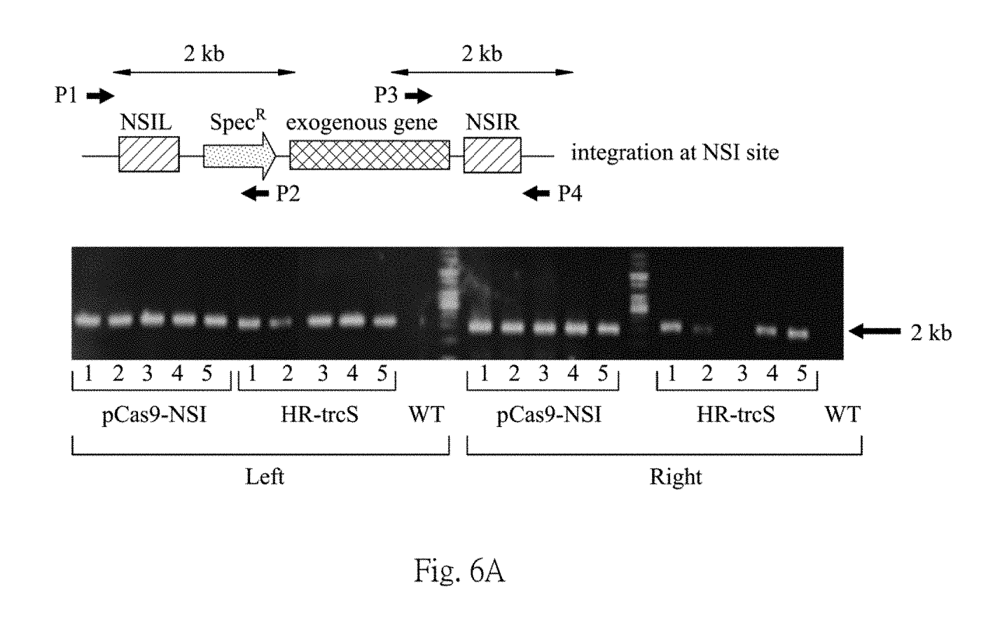

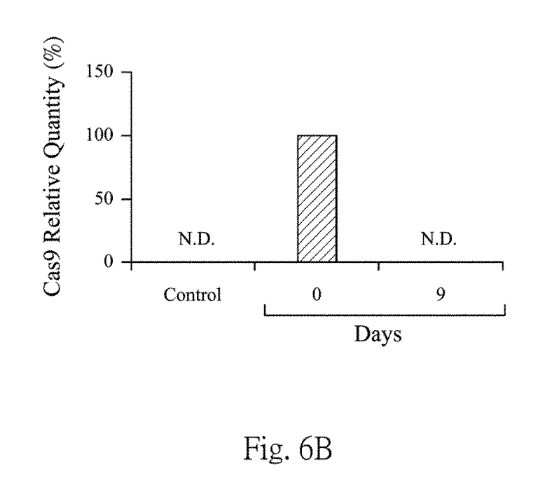

Further, a colony PCR is used in this example to confirm whether the exogenous gene integrated into a precise site in the chromosome of the S. elongatus PCC 7942. Two pairs of primers (P1/P2 and P3/P4) that span the left and right integration junctions are designed for the colony PCR, and a size of left amplicon and the size of right amplicon are about 2 Kb. The nucleotide sequence of the P1 primer, the P2 primer, the P3 primer and the P4 primer is referenced as SEQ ID NO: 32, SEQ ID NO: 33, SEQ ID NO: 34 and SEQ ID NO: 35 respectively. FIG. 6A shows analytical results of the colony PCR for confirming precise integration of the exogenous gene in the chromosome of the S. elongatus PCC 7942. In FIG. 6A, one colony in the HR-trcS group (the conventional method) does not produce the right amplicon; it indicates that the colony does not integrate the exogenous gene precisely. By contrast, all five randomly picked colonies in the Cas9-NSI group contain the correctly integrated cassette; it indicates that the gene editing system of the S. elongatus PCC 7942 of the present disclosure can indeed insert the exogenous gene into the precise site in the chromosome of the S. elongatus PCC 7942.

To demonstrate that the CRISPR/Cas9 expression plasmids will self-disappear after the transformation, a qPCR (quantitative real-time PCR) assay is used by using QCas9 F primer and QCas9 R primer to examine copy number of the Cas9 gene in the transformant at 0 day (immediately after the transformation) and 9 days post-transformation, wherein the nucleotide sequence of the QCas9 F primer and the QCas9 R primer is referenced as SEQ ID NO: 53 and SEQ ID NO: 54 respectively. Further, the copy number of the Cas9 gene in a wild type S. elongatus PCC 7942 is also detected as the control. The copy number of the Cas9 gene at day 0 is used as a baseline to calculate a relative quantification. FIG. 68 shows analytical results of a qPCR for confirming the residual CRISPR/Cas9 expression plasmid of the present disclosure after the transformation, wherein the N.D. (not detectable) represents that a relative copy number of the Cas9 gene is not detected. In FIG. 6B, the copy number of the Cas9 gene in the transformant drops to the baseline level after 9 days post-transformation, attesting that the CRISPR/Cas9 expression plasmid only transiently exists in the transformant.

### 1.4 Optimization of Plasmid Doses for the Homologous Recombination in the S. elongatus PCC 7942

To optimize and verify that the gene editing system of the S. elongatus PCC 7942 of the present disclosure can enhance the homologous recombination efficiency of the exogenous gene and ultimate capacity in the S. elongatus PCC 7942, the pCas9-NSI plasmid and the pHR-trcS plasmid are co-transformed at different doses to find out the optimal plasmid dose. The pCas9-NSI plasmid at 250 ng, 500 ng, 1000 ng and 2000 ng and the pHR-trcS plasmid at 250 ng, 500 ng, 1000 ng and 2000 ng are co-transformed into the S. elongatus PCC 7942 cell respectively, and the homologous recombination efficiency is observed.

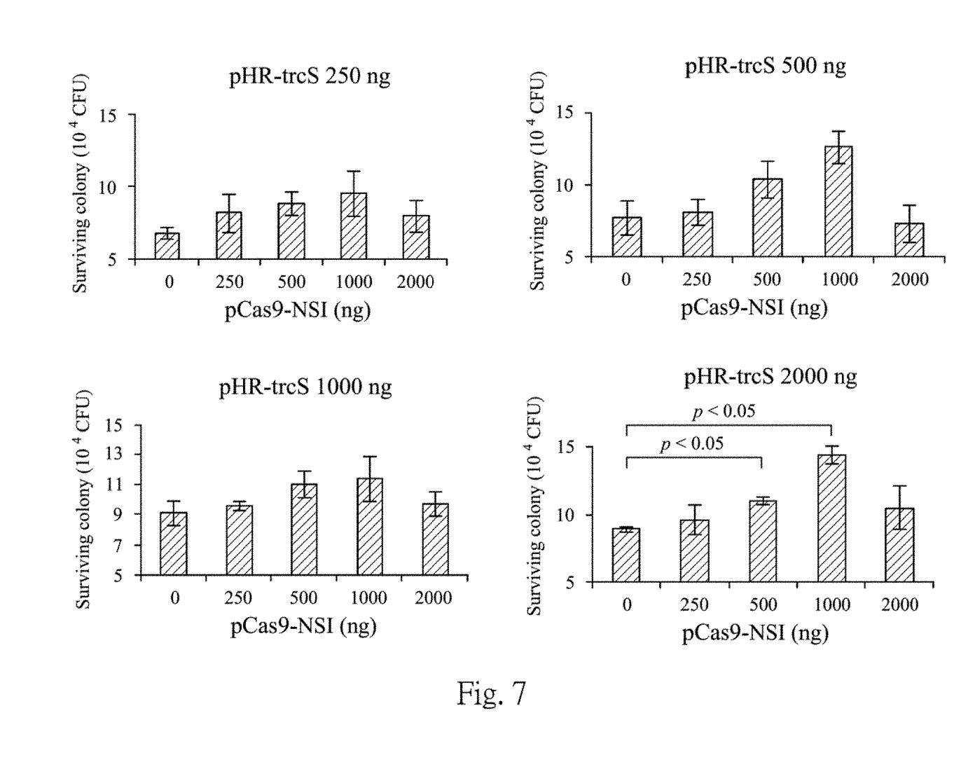

FIG. 7 shows analytical results of homologous recombination efficiency at different dose combinations of the CRISPR/Cas9 expression plasmid and the template plasmid of the gene editing system of the S. elongatus PCC 7942 co-transformed into the S. elongatus PCC 7942 cell. There is almost no promoting effect on homologous recombination efficiency at 250 ng of the pCas9-NSI plasmid. However, homologous recombination efficiency is increased in a dose dependent manner by increasing the dose of the pCas9-NSI plasmid to 500 ng. Among all these doses, 2000 ng of the pHR-trcS plasmid and 1000 ng of the pCas9-NSI plasmid conferr the highest CFU (1.4±0.3×10⁵ CFU), which is 57% (p<0.05) higher than transformation with 2000 ng of the pHR-trcS plasmid (8.9±2.2×10⁴ CFU), a template dose often used for homologous recombination in cyanobacteria. As such, the gene editing system of the S. elongatus PCC 7942 of the present disclosure can improve the homologous recombination efficiency up to 57%.

In the dose of the pHR-trcS plasmid, the homologous recombination efficiency of the transformant transformed with 250 ng of the pHR-trcS plasmid and the pCas9-NSI plasmid (500 ng or 1000 ng) is comparable to that using 2000 ng of the pHR-trcS plasmid. These data indicate that the gene editing system of the S. elongatus PCC 7942 of the present disclosure is able to reduce the amount of the template plasmid to achieve comparable homologous recombination efficiency compared to the conventional method (adding 2000 ng of the pHR-trcS plasmid).

In addition, the lengths of homology arms on the template plasmid are shortened to examine whether the gene editing system of the S. elongatus PCC 7942 of the present disclosure can promote the homologous recombination efficiency for the use of shorter homology arms.

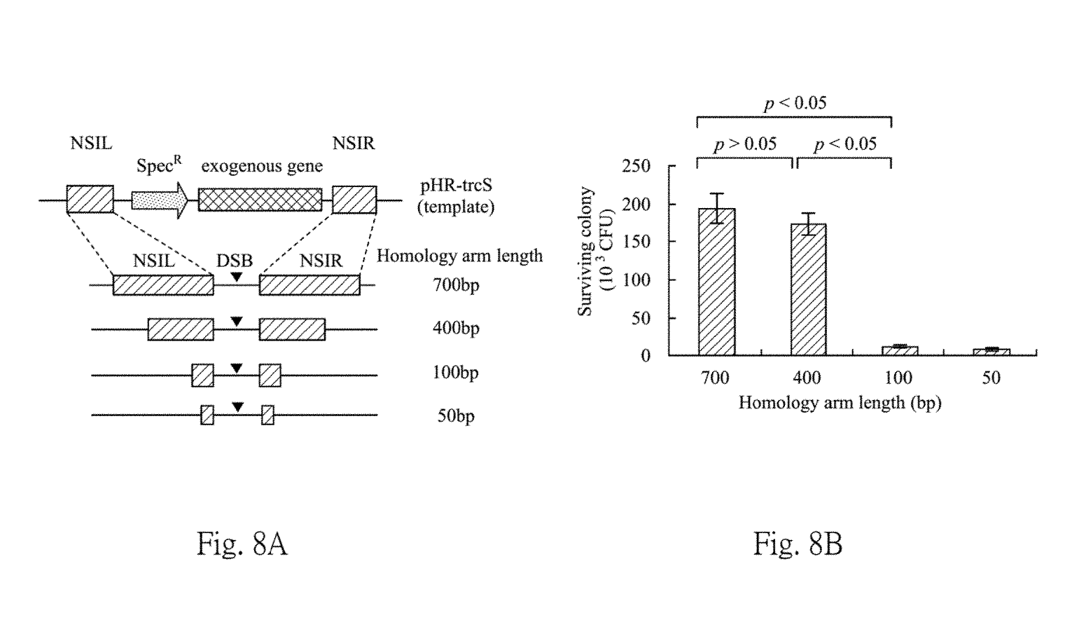

FIG. 8A is a schematic view showing a construction of the template plasmids with different homology arm lengths. A series of plasmids are constructed based on the template plasmid constructed previously (the pHR-trcS plasmid having the left homology arm and the right 700 bp in the length respectively). The plasmids has the same gene expression cassette with the pHR-trcS plasmid, but the lengths of homology arms on the plasmids are shortened from 700 bp to 400 bp, 100 bp or 50 bp. The nucleotide sequence of the 400 bp left homology arm and the 400 bp right homology arm is referenced as SEQ ID NO: 36 and SEQ ID NO: 37 respectively. The nucleotide sequence of the 100 bp left homology arm and the 100 bp right homology arm is referenced as SEQ ID NO: 38 and SEQ ID NO: 39 respectively. The nucleotide sequence of the 50 bp left homology arm and the 50 bp right homology arm is referenced as SEQ ID NO: 40 and SEQ ID NO: 41 respectively. The new template plasmids are co-transformed with the pCas9-NSI plasmid into the S. elongatus PCC 7942, and then observing the homologous recombination efficiency.

FIG. 88 is an analytical tivlyult showing the homologous recombination efficiency of the gene editing system of the S. elongatus PCC 7942 with different homology arm lengths according to one embodiment of the present disclosure. In FIG. 8B, shortening the homology arm length to 400 bp conferrs statistically similar (p>0.05) homologous recombination efficiency when compared with 700 bp, but reducing the homology arm length to 100 or 50 bp leads to precipitously dropped homologous recombination efficiency. These results indicate that the gene editing system of the S. elongatus PCC 7942 of the present disclosure allows for the use of the shorter homology arm without affecting the homologous recombination efficiency.

### 1.5 the CRISPR/Cas9 Gene Editing System Promotes the Homologous Recombination Efficiency in the S. elongatus PCC 7942

One challenge to genome engineering of the S. elongatus PCC 7942 is the oligoploidy nature. The S. elongatus PCC 7942 possesses an average of 4 copies of identical chromosome during the non-growing period. The conventional homologous recombination method does not guarantee the exogenous gene integration into all chromosomes (only integrated into 1-2 copies of the chromosomes as heterozygous recombinants). The heterozygous recombinants will gradually lose the exogenous gene in the continued culture, thus requiring continuous streaking with increasing an antibiotic concentration for more than three weeks selection to integrate the exogenous gene into all chromosomes and become homologous recombinants by giving selection pressure of the antibiotics.

To assess whether the gene editing system of the S. elongatus PCC 7942 can accelerate the selection of homologous recombinants (all chromosomes containing the exogenous genes), the pCas9-NSI plasmid and the pHR-trcS plasmid are co-transformed into the S. elongatus PCC 7942 cell. The transformant is plated on the plate and picked 5 colonies as the test (Cas9-NSI group). The pHR-trcS plasmid alone is transformed into the S. elongatus PCC 7942, and then the transformant is plated on the plate and picked colonies as the control (HR-trcS group). The colonies are cultured in the tube to stationary phase (OD₇₃₀=2.0), and chromosomal DNA is extracted to analyze the copy number of the exogenous gene by qPCR.

This example further discusses whether the gene editing system of the S. elongatus PCC 7942 of the present disclosure can reduce the time spent on knocked out the gene on the chromosome of the S. elongatus PCC 7942. The transformants (the Cas9-NSI group and the HR-trcS group) are re-plated on the agar plates with increasing spectinomycin concentration. The colonies are picked and re-cultured in tube, and then the copy number of the exogenous gene in each transformant is analyzed. The passage process is repeated three times. Moreover, to test whether repeating a CRISPR cleavage of the chromosome can accelerate the selection of the homologous recombinants, the transformants are taken form the tube and then transformed with the pCas9-NSI plasmid to break the chromosomes without the exogenous gene integration in the second and third passage. Aforementioned transformants are plated and picked the colonies. The picked colonies are re-cultured in tube to the stationary phase, and then the chromosomal DNA is extracted to analyze the copy number of the exogenous gene by qPCR (rCas9-NSI group) with qPCR150 F primer and qPCR150 R primer, wherein the nucleotide sequence of the qPCR150 F primer and the qPCR150 R primer is referenced as SEQ ID NO: 55 and SEQ ID NO: 56, respectively.

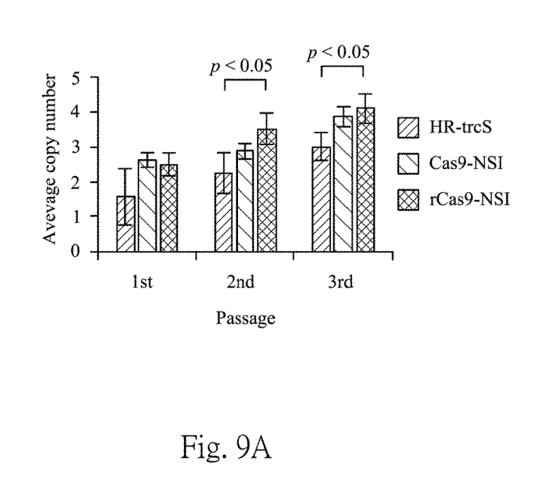

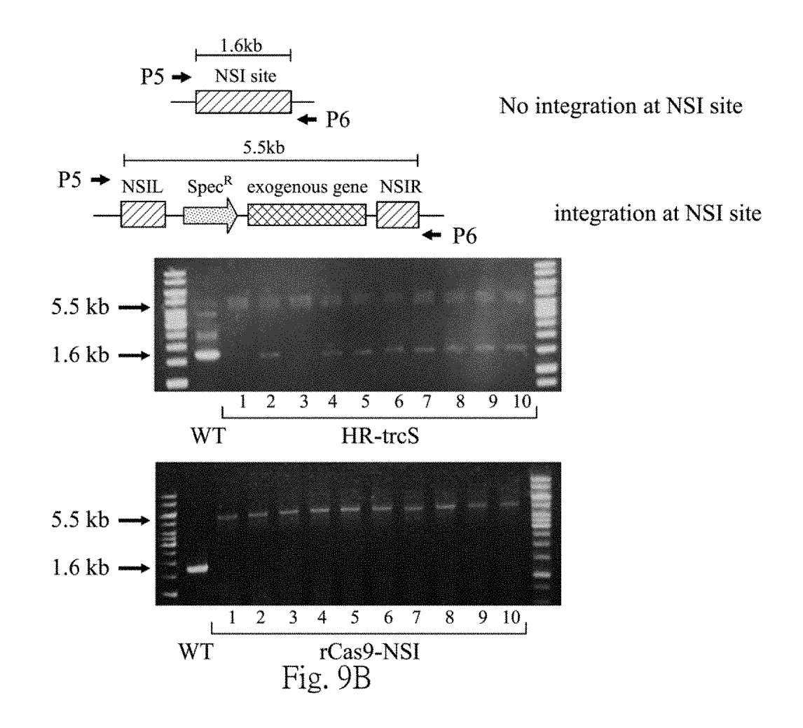

FIG. 9A is an analytical result showing average copy number of the chromosome of the S. elongatus PCC 7942 which is integrated the exogenous gene. The qPCR analysis reveals that in the HR-trcS group the average gene copy number per cell is only 1.60.8 in the first passage and gradually increased to 3.0±0.4 at the third passage. It shows that the conventional method can not convert the transformant into the homologous recombinants even after three subcultures and increasing the antibiotic concentration for the selection. In the Cas9-NSI group, the average copy number per cell reaches 2.6±0.2 in the first passage and increases to 3.9±0.3 in the third passage. The average copy number per cell in the rCas9-NSI group reaches 3.550.5 at the second passage and increases to 4.1±0.4 at the third passage. These data attest that the gene editing system of the S. elongatus PCC 7942 of the present disclosure can accelerate the process for obtaining the homologous recombinants.

To verify the full segregation, 10 colonies are picked from the HR-trcS group and the rCas9-NSI group after 3 passages. Then the colonies are performed the colony PCR by using P5 primer and P6 primer to confirm whether the existence of the NSI site, wherein the nucleotide sequence of the P5 primer and P6 primer is referenced as SEQ ID NO: 42 and SEQ ID NO: 43 respectively. The S. elongatus PCC 7942 possesses 4 copies of the identical chromosomes. If the exogenous gene is completely integrated into 4 copies of the chromosomes, a NSI site signal (1.6 kb) will not be detected. FIG. 96 shows analytical results of a colony PCR for confirming integration of the exogenous gene in all of the chromosome of the S. elongatus PCC 7942 by the gene editing system of the S. elongatus PCC 7942 according to one embodiment of the present disclosure. In FIG. 9B, the NSI site signal (1.6 kb) is detected in almost every colony of the HR-trcS group, whereas the NSI site signal is not detected in the rCas9-NSI group. It indicates that the gene editing system of the S. elongatus PCC 7942 of the present disclosure can effectively accelerate the speed of homologous recombination.

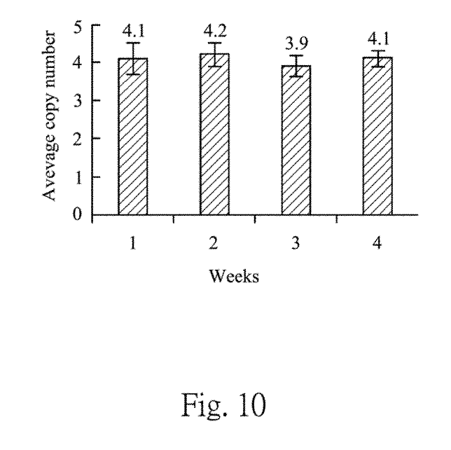

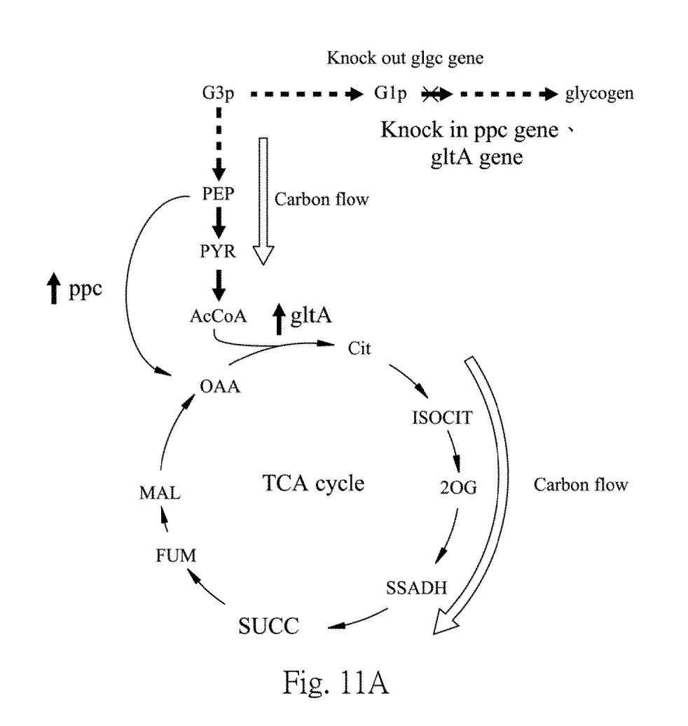

To attest that the homologous recombinant remains stable, one colony is picked from the rCas9-NSI group and re-cultured in a shake flask without spectinomycin for 4 weeks the tube for four weeks. The cells are subcultured and sampled every week for the qPCR analysis. FIG. 10 is an analytical result showing a stability of an integration of the exogenous gene in the chromosome of the S. elongatus PCC 7942. In FIG. 10, the result demonstrates that the average gene copy number is 4.1±0.4 at week 1 and remains 4.1±0.2 at week 4, confirming that the number of the exogenous gene in the S. elongatus PCC 7942 remains stable.

### 1.6 Engineering the Metabolic Pathway of the S. elongatus PCC 7942 by the CRISPR/Cas9 Gene Editing System

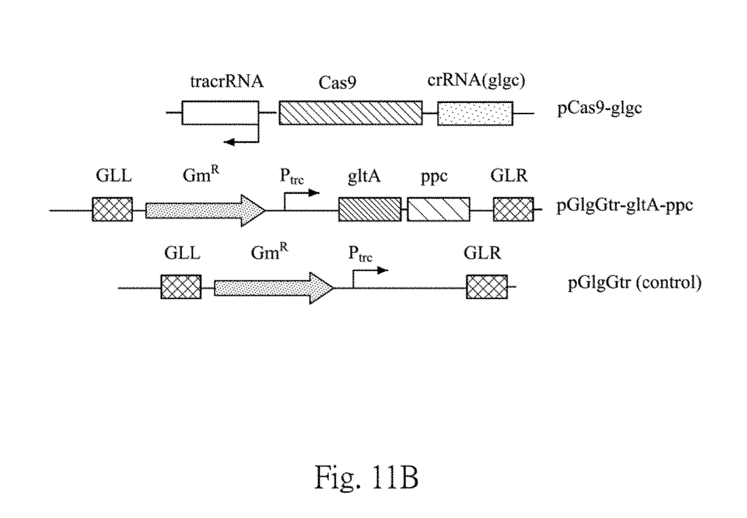

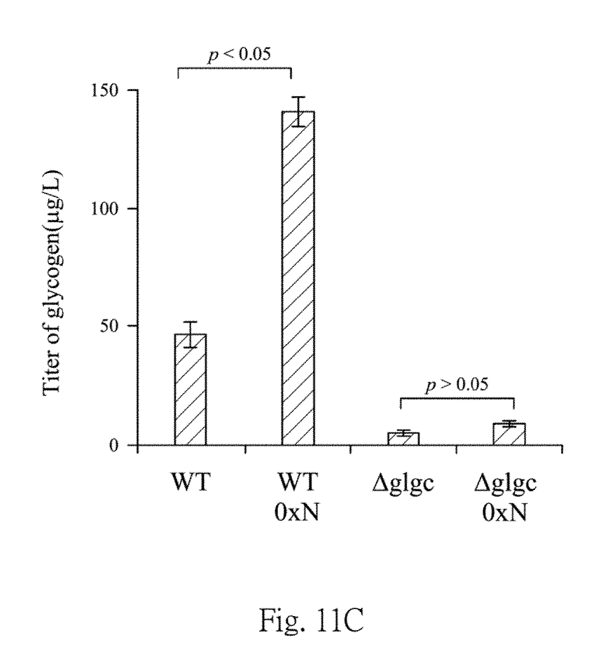

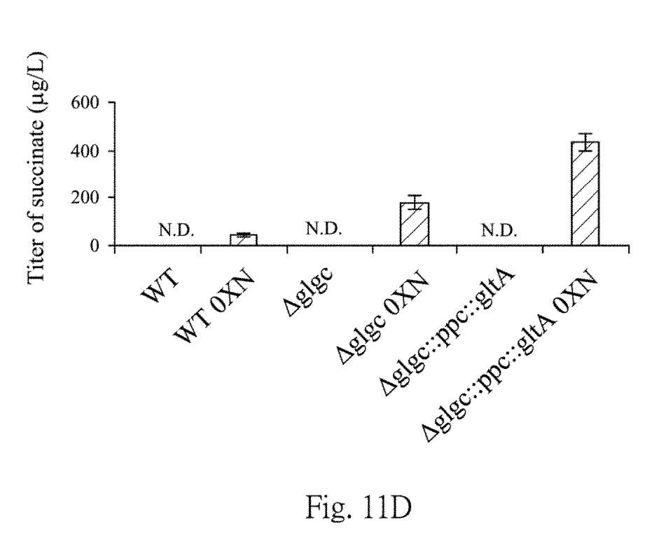

This example further discusses whether the gene editing system of the S. elongatus PCC 7942 of the present disclosure can be used for editing the metabolic pathway of the S. elongatus PCC 7942. FIG. 11A is a schematic view showing metabolic pathways and regulatory genes of the S. elongatus PCC 7942. FIG. 11B is a schematic view showing a construction of a plasmid for knocking out a glgc gene of the chromosome of the S. elongatus PCC 7942 by the gene editing system. FIG. 11C is an analytical result showing a change of glycogen accumulation in the S. elongatus PCC 7942 knocked out the glgc gene. FIG. 11D is an analytical result showing a change of succinate (SUCC) production in the S. elongatus PCC 7942 knocked out the glgc gene.

To construct the S. elongatus PCC 7942 having ability to produce succinate, the gene editing system of the S. elongatus PCC 7942 is used to knock out the glgc gene and knock in the gltA gene and the ppc gene into the S. elongatus PCC 7942 cell. A pCas9-glgc plasmid, a pGlgGtr-gltA-ppc plasmid and a pGlgGtr plasmid are constructed. The pCas9-glgc plasmid is constructed based on the CRISPR/Cas9 expression plasmid constructed previously, and the nucleotide sequence of the crRNA of the pCas9-glgc plasmid is referenced as SEQ ID NO: 44 and modified as the sequence targeting the glgc gene, wherein the glgc gene is a gene that produces glycogen. The pGlgGtr-gltA-ppc plasmid and the pGlgGtr plasmid are the template plasmids, wherein the homology arm of the pGlgGtr-gltA-ppc plasmid is homologous to partial sequence of the glgc gene and the nucleotide sequence of the left homology arm and the right homology arm of the pGlgGtr-gltA-ppc plasmid is referenced as SEQ ID NO: 45 and SEQ ID NO: 46 respectively. The pGlgGtr-gltA-ppc plasmid further includes gentamycine resistance gene having the nucleotide sequence referenced as SEQ ID NO: 47 and two exogenous genes, the gltA gene and the ppc gene, wherein the gltA gene and the ppc gene initiated by a trc promoter can increase the carbon source into the TCA cycle to increase the succinate production. The nucleotide sequence of the trc promoter, the gtA gene and the ppc gene is referenced as SEQ ID NO: 48, SEQ ID NO: 49 and SEQ ID NO: 50 respectively. The pGlgGtr plasmid is the template plasmid of the control. The homology arm of the pGlgGtr plasmid is also homologous to partial sequence of the glgc gene, but the pGlgGtr plasmid only includes gentamycine resistance gene without the exogenous gene. The nucleotide sequence of the left homology arm and the right homology arm of the pGlgGtr plasmid is referenced as SEQ ID NO: 51 and SEQ ID NO: 52 respectively.

The pCas9-glgc plasmid and the template plasmid (the pGlgGtr-gltA-ppc plasmid or the pGlgGtr plasmid) are co-transformed into the S. elongatus PCC 7942 cell. Then colonies are picked and re-cultured under nitrogen starvation conditions to analyze production changes of the glycogen and the succinate. In FIG. 11C, WT represents the wild type S. elongatus PCC 7942, Δglgc represents the S. elongatus PCC 7942 knocked out the glgc gene, and 0×N represents the culture under the nitrogen starvation condition. Under the nitrogen starvation condition, a titer of the glycogen is 140.5±6.1 ug/L in the wild type S. elongatus PCC 7942, whereas the titer of glycogen reduces to 9±1.2 ug/L in the S. elongatus PCC 7942 knocked out the glgc gene. It indicates that the gene editing system of the S. elongatus PCC 7942 of the present disclosure can rapidly knock out the glgc gene to drop the production of the glycogen. In FIG. 11D, the WT represents the wild type S. elongatus PCC 7942, the Δglgc represents the S. elongatus PCC 7942 knocked out the glgc gene, Δglgc::ppc::gltA represents the S. elongatus PCC 7942 knocked out the glgc gene and knocked in the gltA gene and the ppc gene, the 0×N represents the culture under nitrogen starvation condition, and N.D. represents that the succinate is not detected. Under the nitrogen starvation condition, a titer of the succinate is 40.5±6.6 ug/L in the wild type S. elongatus PCC 7942, whereas the titer of the succinate increases about 10 times to 435±35 ug/L in the S. elongatus PCC 7942 knocked out the glgc gene and knocked in the gltA gene and the ppc gene. It indicates that the gene editing system of the S. elongatus PCC 7942 of the present disclosure can knock out the glgc gene and knock in the gltA gene and the ppc gene to promote carbon flow into the TCA cycle and improve the succinate production.

Under nitrate-replete conditions, both WT and recombinant cells accumulated low levels of glycogen (FIG. 5C) and negligible levels of succinate (FIG. 5D). Under nitrogen starvation conditions (0×N), significantly increased glycogen accumulation was observed in the WT cells, but not in the Δglgc group (FIG. 5C). The phenotype change (Fig. S4) further confirmed the knockout of glgc. Such glgc knockout resulted in an increased succinate production to 486.3±63.7 μg/L/OD730 (FIG. 5D). Integration of gltA and ppc into the glgc locus further enhanced the succinate titer to 707.0±53.8 μg/L/OD730, a ≈17-fold increase when compared with that of the WT cells (40.5±6.6 μg/L/OD730) (FIG. 5D). These data collectively demonstrated that the CRISPR-Cas9-mediated gene knock-out/knock-in was able to regulate the metabolic pathway in PCC 7942 and improve succinate production.

To sum up, the gene editing system of the S. elongatus PCC 7942 of the present disclosure and the gene editing method of the S. elongatus PCC 7942 of the present disclosure can effectively and simultaneously trigger programmable double strand breaks at the target gene on four copies of the chromosome of the S. elongatus PCC 7942 to cause the death of the S. elongatus PCC 7942 cell. The exogenous gene can be precisely integrated into the genome of the S. elongatus PCC 7942 by transforming the template plasmid, and the Cas9 gene and the crRNA of the template plasmid are not detectable on 9 days after the transformation. In addition, the double strand break caused by the gene editing system of the S. elongatus PCC 7942 of the present disclosure imposes an intrinsic selective pressure on the S. elongatus PCC 7942 and hence improving the homologous recombination efficiency as well as allowing for the use of lower amount of the template plasmid and shorter homology arms. Further, the double strand break caused by the gene editing system of the S. elongatus PCC 7942 of the present disclosure enhances changes of concomitant integration of the exogenous gene into all chromosomes of the S. elongatus PCC 7942, thereby accelerating a process of obtaining stable and homogenous recombinant strains, which stably express the exogenous gene. Also, the gene editing system of the S. elongatus PCC 7942 of the present disclosure enables the simultaneous and precise gene knock-out and knock-in so as to improve the succinate production in the S. elongatus PCC 7942.

### II. A Gene Expression Interference System of the S. elongatus PCC 7942 of the Present Disclosure

### 2.1 Fluorescence Expression Systems with Different Promoters

To date, expression intensities of different kinds of promoters in the S. elongatus PCC 7942 are not well known. To establish the gene expression interference system of the S. elongatus PCC 7942 of the present disclosure in the S. elongatus PCC 7942, promoter activities of various promoters in the S. elongatus PCC 7942 are compared in this example.

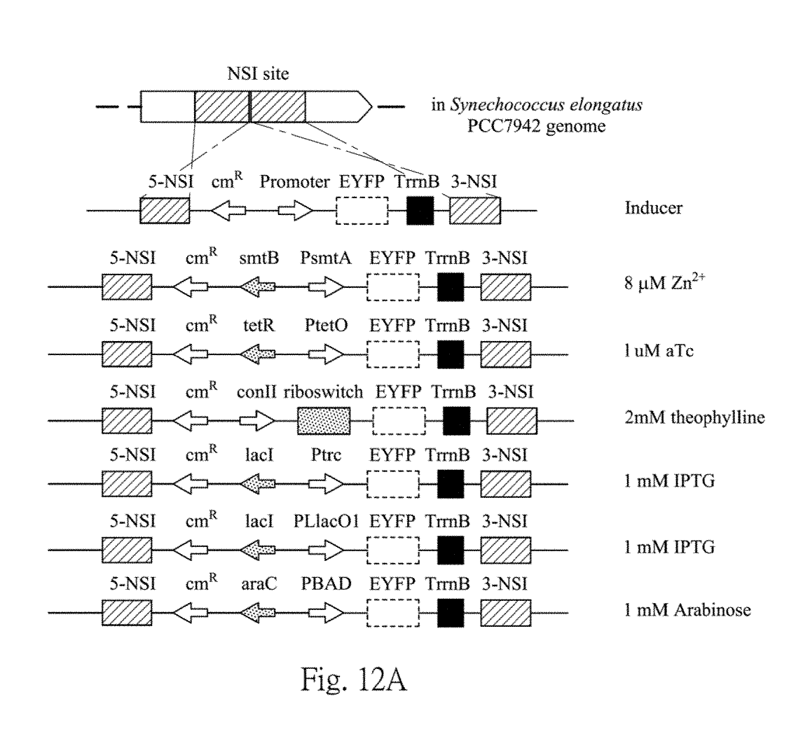

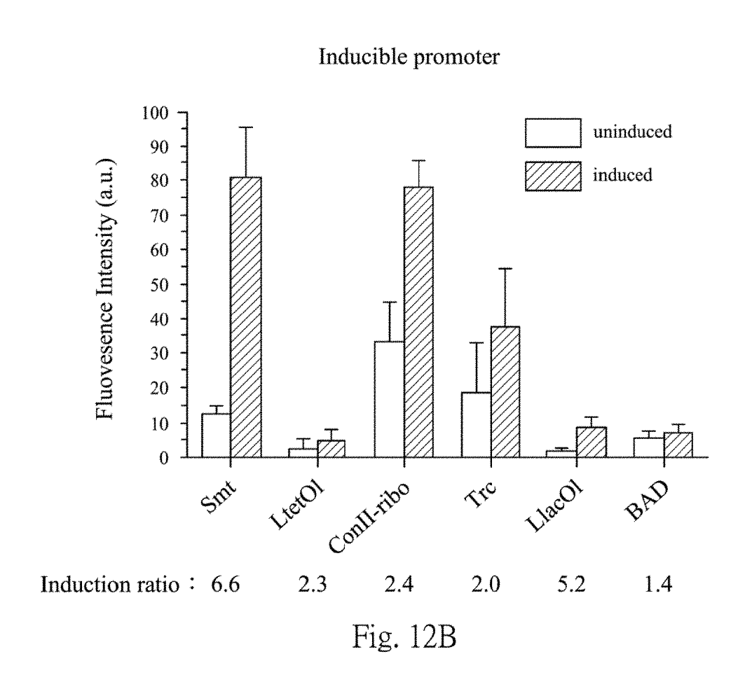

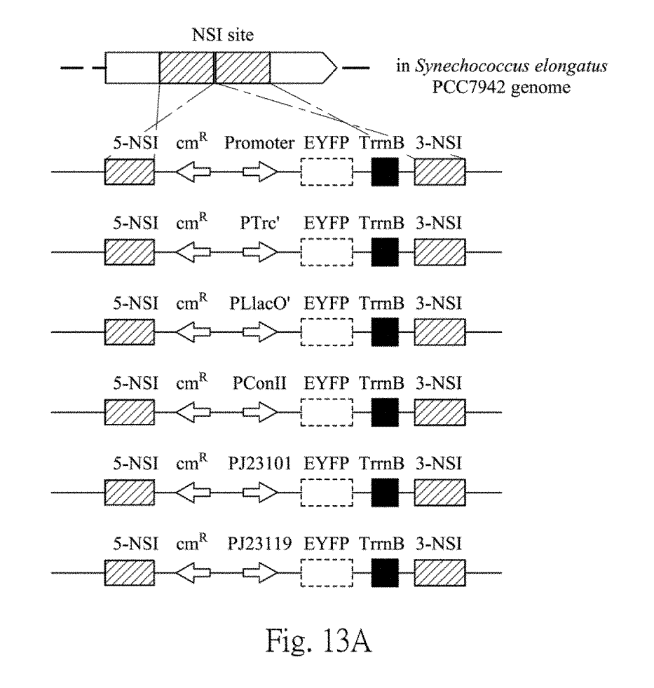

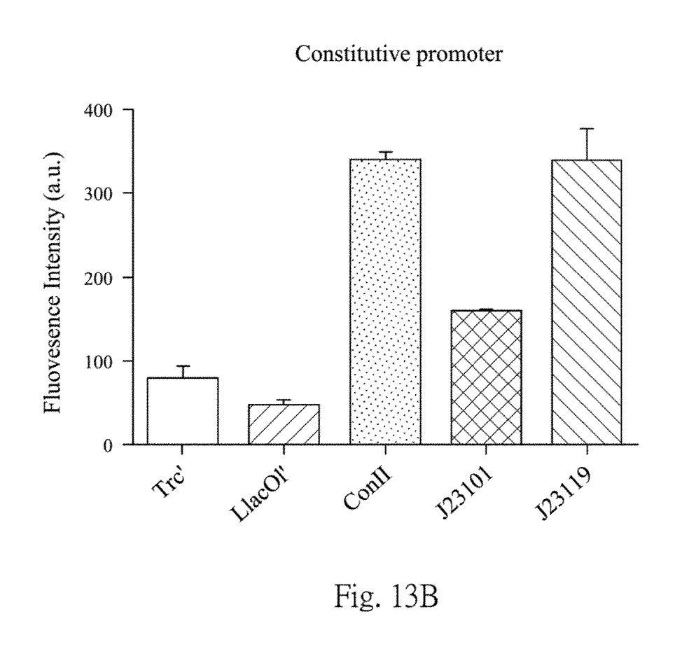

The promoters can be generally divided into inducible promoters and constitutive promoters. FIG. 12A is a schematic view showing constructions of the inducible promoters of the gene expression interference system of the S. elongatus PCC 7942 according to yet another embodiment of the present disclosure. FIG. 12B shows analytical results of the inducible promoters of the gene expression interference system of the S. elongatus PCC 7942. FIG. 13A is a schematic view showing constructions of the constitutive promoters of the gene expression interference system of the S. elongatus PCC 7942 according to yet another embodiment of the present disclosure. FIG. 13B shows analytical results of the constitutive promoters of the gene expression interference system of the S. elongatus PCC 7942.

A yellow fluorescent protein (eyfp) gene is used as a reporter gene in this example, and various promoters is inserted in front of the eyfp gene respectively. Further, all of the constructed plasmid include the homology region which is homologous to the NSI gene of the S. elongatus PCC 7942 and the chloramphenicol resistance (Cm^{R}) gene. The inducible promoters used in this example are a Smt promoter, a LtetO1 promoter, a ConII-ribo promoter, a Trc promoter, a LlacO1 promoter and a BAD promoter, wherein inducers of aforementioned inducible promoters is 8 μM of Zn²⁺, 1 μM of aTc, 2 mM of theophylline, 1 mM of isopropyl β-D-1-thiogalactopyranoside (IPTG), 1 mM of IPTG and 1 mM of arabinose, respectively. The nucleotide sequence of the Smt promoter is referenced as SEQ ID NO: 8, the nucleotide sequence of the LtetO1 promoter is referenced as SEQ ID NO: 9, the nucleotide sequence of the ConII-ribo promoter is referenced as SEQ ID NO: 10, the nucleotide sequence of the Trc promoter is referenced as SEQ ID NO: 11, the nucleotide sequence of the LlacO1 promoter is referenced as SEQ ID NO: 12, and the nucleotide sequence of the BAD promoter is referenced as SEQ ID NO: 13. The constitutive promoters used in this example are a Trc′ promoter, a LlacO1′ promoter, a ConII promoter, a J23101 promoter and a J23119 promoter. The nucleotide sequence of the Trc′ promoter is referenced as SEQ ID NO: 14, the nucleotide sequence of the LlacO1′ promoter is referenced as SEQ ID NO: 15, the nucleotide sequence of the ConII promoter is referenced as SEQ ID NO: 16, the nucleotide sequence of the J23101 promoter is referenced as SEQ ID NO: 17, and the nucleotide sequence of the J23119 promoter is referenced as SEQ ID NO: 18.

Constructed plasmids are natural transformed into the S. elongatus PCC 7942 cells, and then the S. elongatus PCC 7942 cells are streaked onto BG-11 agar plates containing chloramphenicol and cultured for 7-9 days. After the cultivation, the colonies at the amount of an inoculating loop are scraped and cultured in 20 mL of BG-11 medium containing the chloramphenicol. The inducer is added into the inducible promoter group at OD₇₃₀ of 0.6-0.8, while the constitutive promoter group does not add any inducer. After another 24 hours cultivation (OD₇₃₀ of 1-1.5), fluorescence intensities of the inducible promoters and the constitutive promoters are analyzed by a flow cytometer.

As shown in FIGS. 12B and 13B, the Smt promoter induced by Zn²⁺ has the highest induction rate (6.6 fold) and the highest fluorescence expression level (80.3 a.u.) after an induction in the inducible promoter group. The induction rate and the fluorescence expression level of the other inducible promoters are 2.3 fold and 4.7 a.u. in the LtetO1 promoter, 2.4 fold and 77.9 a.u. in the ConII-ribo promoter, 2 fold and 37.7 a.u. in the Trc promoter, 5.2 fold and 8.6 a.u. in the LlacO1 promoter, and 1.4 fold and 7.0 a.u. in the BAD promoter.

In the constitutive promoter group, the fluorescence intensity is in order of the ConII promoter≈the PJ23119 promoter>the J23101 promoter>the Trc′ promoter>the LlacO1′ promoter, and the fluorescence expression level is 339 a.u., 338 a.u., 158 a.u., 77 a.u. and 46 a.u., respectively. From the above results, the Smt promoter has the highest induction rate (6.6 fold) and the highest fluorescence expression level after the induction in the S. elongatus PCC 7942, and the ConII promoter and the J23119 promoter are the promoters having the best constitutive expression. Therefore the Smt promoter, the ConII promoter and the J23119 promoter are choose for establish the gene expression interference system of the S. elongatus PCC 7942 of the present disclosure in subsequent examples. However, to maximize the yield of the target product by expressing an exogenous protein gene in the S. elongatus PCC 7942, it not merely needs to overexpress the exogenous protein gene but needs to consider the induction rate and an extent of the gene expression. Hence not only the promoter having the strongest expression is optimal, but the other promoters can be used in the gene expression interference system of the S. elongatus PCC 7942 of the present disclosure to regulate the extent of the gene expression for optimizing the yield of the target product.

### 2.2 Establishment of CRISPRi Gene Expression Interference System of the S. elongatus PCC 7942

The gene expression interference system of the S. elongatus PCC 7942 of the present disclosure includes the S. elongatus PCC 7942 cell, a dCas9 expression plasmid and a sgRNA plasmid.

The dCas9 expression plasmid successively includes a first left homology arm, a first promoter, a dCas9 gene, a first antibiotic resistance gene and a first right homology arm, wherein the first left homology arm and the first right homology arm compose a first homology region. According to one embodiment of this example, the dCas9 expression plasmid is a pSdCas9-CY′ plasmid, and a yellow fluorescent protein is used as a test model. There is no the yellow fluorescent protein in the S. elongatus PCC 7942 cell, hence an eyfp gene existed in the pSdCas9-CY is used as the target gene. Further, the constitutive promoter is integrated in front of the eyfp gene to initiate a transcription of the eyfp gene. The nucleotide sequence of the first left homology arm of the pSdCas9-CY plasmid is referenced as SEQ ID NO: 19. The first promoter in this example is the Smt promoter, which is the inducible promoter and the nucleotide sequence of the Smt promoter is referenced as SEQ ID NO: 8. The nucleotide sequence of the dCas9 gene is referenced as SEQ ID NO: 20. The constitutive promoter in front of the eyfp gene is the ConII promoter with the nucleotide sequence referenced as SEQ ID NO: 16. The nucleotide sequence of the eyfp gene is referenced as SEQ ID NO: 21. The first antibiotic resistance gene is the Cm^{R} gene with the nucleotide sequence referenced as SEQ ID NO: 22. The nucleotide sequence of the first right homology arm is referenced as SEQ ID NO: 23. The first homology region composed of the first left homology arm and the first right homology arm is partial sequence of the NSI gene of the S. elongatus PCC 7942 cell. The first left homology arm, the Smt promoter, the dCas9 gene, the ConII promoter, the eyfp gene, the Cm^{R} gene and the first right homology arm are constructed on pdCas9 vector (Addgene) to obtain the pSdCas9-CY′ plasmid. Therefore, the Smt promoter of the pSdCas9-CY′ plasmid can regulate the expression of the dCas9 gene, the ConII promoter can initiate the expression of the eyfp gene, and the expression of the Cm^{R} gene is initiated by another promoter of the antibiotic resistance gene.

The sgRNA plasmid successively includes a second left homology arm, a second promoter, a sgRNA, a second antibiotic resistance gene and a second right homology arm, wherein the second left homology arm and the second right homology arm compose a second homology region, a sequence of the sgRNA is homologous to a sequence of a target gene, the target gene is on a chromosome of the S. elongatus PCC 7942 or on an exogenous plasmid, the second homology region and the first homology region are different, and the second antibiotic resistance gene and the first antibiotic resistance gene are different. A series of the sgRNA plasmids, a psgRNA::Φ plasmid, a psgRNA::P1 plasmid, a psgRNA::NT1 plasmid, and a psgRNA::NT2 plasmid, are constructed in this example. These sgRNA plasmids differ in the sequence of the sgRNAs, which can target on different positions (P1, NT1, and NT2) on the non-template strand of the target gene expression cassette, and other part of these sgRNA plasmids are the same. The nucleotide sequence of the second left homology arm of the sgRNA plasmid is referenced as SEQ ID NO: 24. The second promoter in this example is the J23119 promoter, which is the constitutive promoter and the nucleotide sequence of the J23119 promoter is referenced as SEQ ID NO: 18. The second antibiotic resistance gene is a kanamycin resistance (Km^{R}) gene with the nucleotide sequence referenced as SEQ ID NO: 25. The nucleotide sequence of the second right homology arm is referenced as SEQ ID NO: 26. The second homology region composed of the second left homology arm and the second right homology arm is partial sequence of the neutral site II (NSII) gene of the S. elongatus PCC 7942 cell. The nucleotide sequence of the sgRNA of the psgRNA::Φ plasmid is referenced as SEQ ID NO: 27, the nucleotide sequence of of the sgRNA of the psgRNA::P1 plasmid is referenced as SEQ ID NO: 28, the nucleotide sequence of the sgRNA of the psgRNA::NT1 plasmid is referenced as SEQ ID NO: 29, and the nucleotide sequence of the sgRNA of the psgRNA::NT2 plasmid is referenced as SEQ ID NO: 30. The second left homology arm, the second promoter, the sgRNA, the second antibiotic resistance gene and the second right homology arm are constructed on NSII_plus vector to obtain a serial of the sgRNA plasmids.

The first promoter in the gene expression interference system of the S. elongatus PCC 7942 not only can be the Smt promoter, but also can be the LtetO1 promoter, the ConII-ribo promoter, the LlacO1 promoter, the BAD promoter, the Trc promoter, the Trc′ promoter, the LlacO1′ promoter, the ConII promoter, the J23101 promoter or the J23119 promoter. The second promoter in the gene expression interference system of the S. elongatus PCC 7942 not only can be the J23119 promoter, but also can be the Smt promoter, the LtetO1 promoter, the ConII-ribo promoter, the LlacO1 promoter, the BAD promoter, the Trc promoter, the Trc′ promoter, the LlacO1′ promoter, the ConII promoter or the J23101 promoter. The first homology region of the dCas9 expression plasmid can be the NSI gene or the NSII gene. The second homology region of the sgRNA plasmid also can be the NSI gene or the NSII gene, but the second homology region differs from the first homology region. The first antibiotic resistance gene of the dCas9 expression plasmid can be the Spec^{R} gene, the Km^{R} gene or the Cm^{R} gene. The second antibiotic resistance gene of the sgRNA plasmid also can be the Spec^{R} gene, the Km^{R} gene or the Cm^{R} gene, but the second antibiotic resistance gene differs from the first antibiotic resistance gene.

### 2.3 A Method for Interfering Gene Expression of S. elongatus PCC 7942

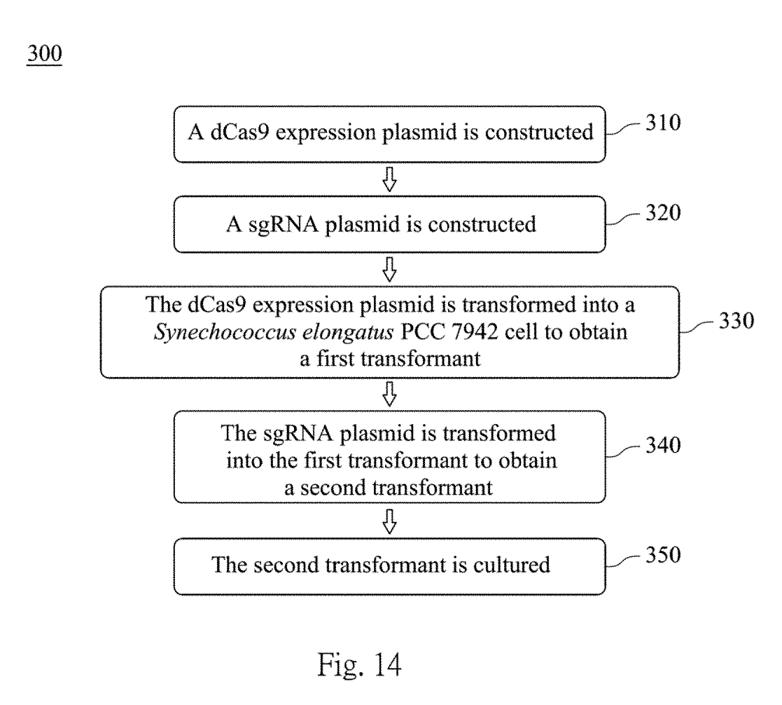

FIG. 14 is a flow diagram showing the method 300 for interfering gene expression of the S. elongatus PCC 7942 according to still another embodiment of the present disclosure. In FIG. 14, the method 300 for interfering gene expression of the S. elongatus PCC 7942 includes a step 310, a step 320, a step 330, a step 340 and a step 350.

In the step 310, the dCas9 expression plasmid is constructed. The dCas9 expression plasmid successively includes the first left homology arm, the first promoter, the dCas9 gene, the first antibiotic resistance gene and the first right homology arm, wherein the first left homology arm and the first right homology arm compose a first homology region.

In the step 320, the sgRNA plasmid is constructed. The sgRNA plasmid successively includes the second left homology arm, the second promoter, the sgRNA, the second antibiotic resistance gene and the second right homology arm, wherein the second left homology arm and the second right homology arm compose a second homology region, a sequence of the sgRNA is homologous to a sequence of a target gene, the target gene is on the chromosome of the S. elongatus PCC 7942 or on an exogenous plasmid, the second homology region and the first homology region are different, and the second antibiotic resistance gene and the first antibiotic resistance gene are different.

In the step 330, the dCas9 expression plasmid is transformed into the S. elongatus PCC 7942 cell to obtain a first transformant, wherein the first homology region of the dCas9 expression plasmid and the first homology region of the chromosome of the first transformant perform a homologous recombination to insert the first promoter, the dCas9 gene and the first antibiotic resistance gene into the first homology region of the chromosome of the first transformant.

In the step 340, the sgRNA plasmid is transformed into the first transformant to obtain a second transformant, wherein the second homology region of the sgRNA plasmid and the second homology region of the chromosome of the second transformant perform the homologous recombination to insert the second promoter, the sgRNA and the second antibiotic resistance gene into the second homology region of the chromosome of the second transformant.

In the step 350, the second transformant is cultured and the inducer is added to induce the dCas9 expression plasmid therein to express a dCas9 protein, wherein the dCas9 protein and the sgRNA expressed from the sgRNA plasmid form a dCas9 protein complex, and then the dCas9 protein complex bind to a target gene to inhibit the expression of the target gene.

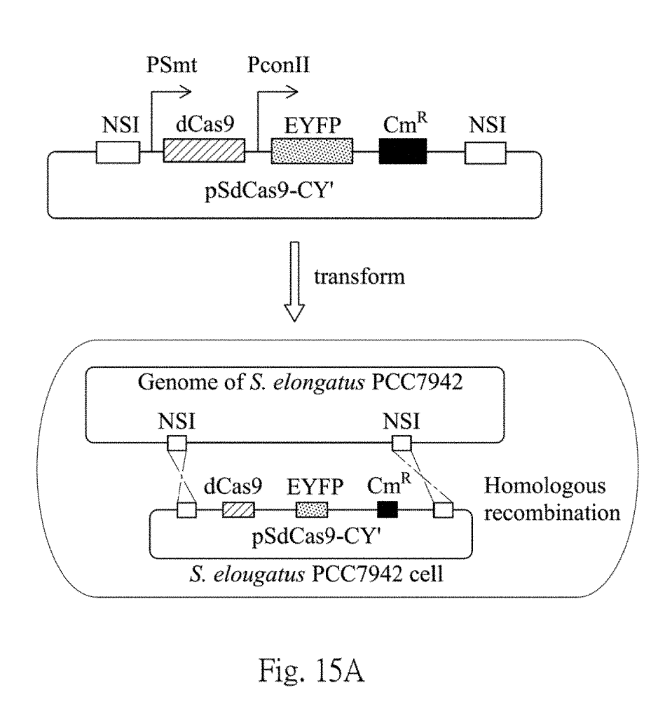

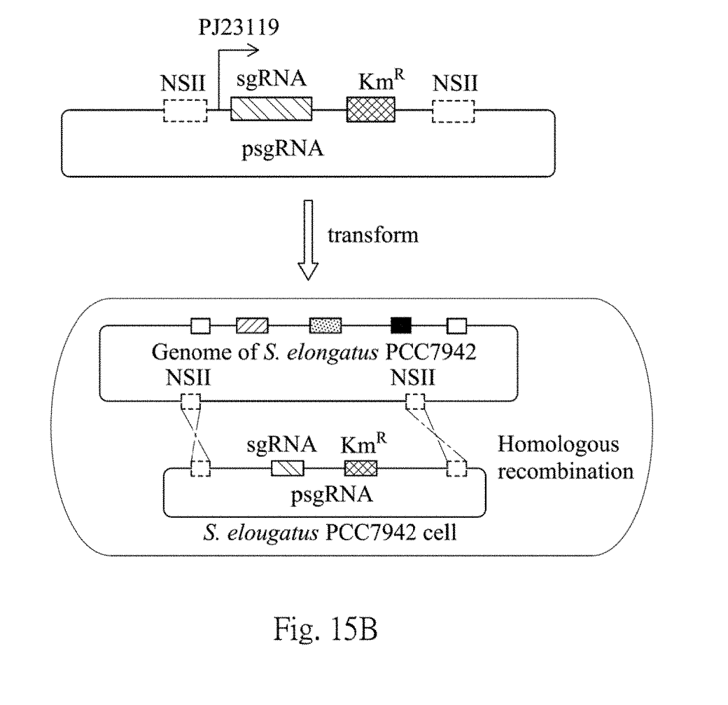

FIG. 15A is a schematic view showing a construction and homologous recombination of a dCas9 plasmid according to still another embodiment of the present disclosure. FIG. 15B is a schematic view showing a construction and homologous recombination of a sgRNA plasmid according to still another embodiment of the present disclosure. According to one embodiment of this example, the dCas9 expression plasmid is the pSdCas9-CY plasmid. The pSdCas9-CY′ plasmid is transformed into the S. elongatus PCC 7942 cell. The first left homology arm and the first right homology arm of the pSdCas9-CY′ plasmid and the NSI gene of the chromosome of the S. elongatus PCC 7942 perform the homologous recombination to insert the Smt promoter, the dCas9 gene, the ConII promoter, the eyfp gene and the Km^{R} gene into the NSI gene of the chromosome of the S. elongatus PCC 7942 to obtain the first transformant. Then the sgRNA plasmid is transformed into the first transformant. The second left homology arm and the second right homology arm of the sgRNA plasmid and the NSII gene of the chromosome of the S. elongatus PCC 7942 perform the homologous recombination to insert the J23119 promoter, the sgRNA and the Cm^{R} gene into the NSII gene of the chromosome of the S. elongatus PCC 7942 to obtain the second transformant.

### 2.4 Effects of the Gene Expression Interference System of the S. elongatus PCC 7942 of the Present Disclosure

To test whether the gene expression interference system of the S. elongatus PCC 7942 of the present disclosure can indeed inhibit the expression of the target gene, the pSdCas9-CY′ plasmid is transformed into the S. elongatus PCC 7942 cell to obtain the first transformant, and then the psgRNA::Φ plasmid, the psgRNA::P1 plasmid, the psgRNA::NT1 plasmid and the psgRNA::NT2 plasmid is transformed into the first transformant respectively to obtain the second transformant (the psgRNA::Φ group, the psgRNA::P1, the psgRNA::NT1 group and the psgRNA::NT2 group). The second transformant can simultaneously express the dCas9 protein, the yellow fluorescent protein and the sgRNA. Then the dCas9 protein and the sgRNA form a dCas9 protein complex for targeting on the ConII promoter or the eyfp gene to inhibit the expression of the yellow fluorescent protein. The the sequence of the sgRNA of the psgRNA::Φ plasmid does not target on any sequence of the chromosome of the S. elongatus PCC 7942.

To confirm that both the dCas9 protein and the sgRNA are required for an inhibitory effect of the gene expression interference system of the S. elongatus PCC 7942 of the present disclosure, a pConII-EYFP plasmid, which does not have the dCas9 gene, is transformed into the S. elongatus PCC 7942 cell and then inserted into the NSI gene of the chromosome of the S. elongatus PCC 7942. The psgRNA::P1 plasmid is transformed into aforementioned S. elongatus PCC 7942 cell and then inserted into the NSII gene of the chromosome of the S. elongatus PCC 7942 (P1 group). Further, there is a dCas9 group transformed with the pSdCas9-CY plasmid and without the sgRNA plasmid into the S. elongatus PCC 7942 cell in this example. The P1 group and the dCas9 group are used as the controls for proving that the dCas9 protein alone or the sgRNA alone can not result in the inhibition of the expression of the yellow fluorescent protein.

After confirming that the transformants are successfully inserted the plasmida into the correct position of the chromosome, the fluorescence intensity of the dCas9::Φ group is compared to the dCas9 group and the P1 group first to confirm that the expression of the dCas9 protein and the sgRNA::Φ do not affect the fluorescence intensity, and both the dCas9 protein and the sgRNA are required in the gene expression interference system of the S. elongatus PCC 7942 of the present disclosure. Because the Smt promoter is found to be unable to regulate the dCas9 protein during the test and causes the constitutive expression, the Zn²⁺ inducer is not added in the subsequent culture. A single colony of the transformant is cultured in the shake flask containing 40 ml of BG-11 medium. The transformant is analyzed by a fluorescence microscope and the flow cytometer at OD₇₃₀ of 1-1.5 to observe the inhibition of the gene expression interference system of the S. elongatus PCC 7942 of the present disclosure.

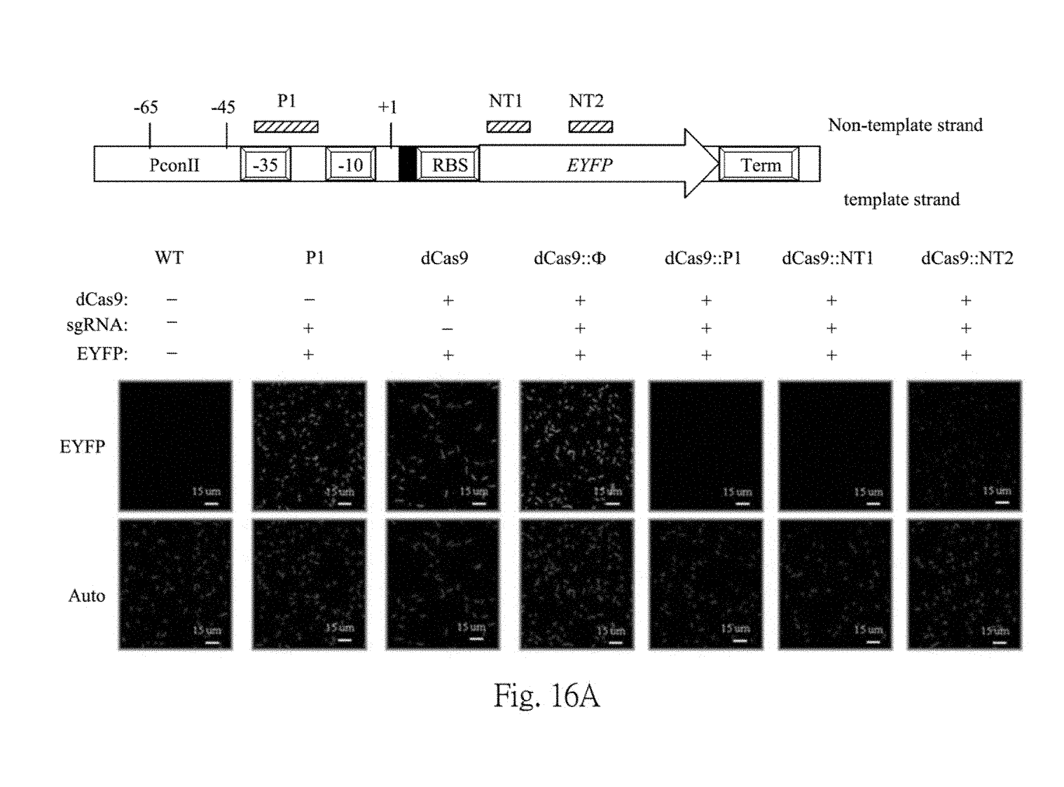

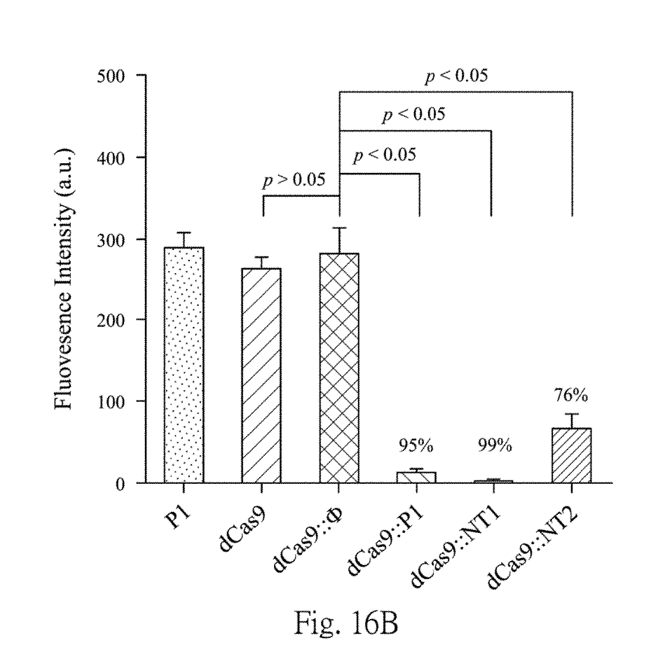

FIGS. 16A and 16B are analytical results showing an expression of a target gene inhibited by the gene expression interference system of the S. elongatus PCC 7942 according to yet another embodiment of the present disclosure, wherein the analytical results of FIG. 16A are observed by the fluorescence microscope, and the analytical results of FIG. 16B are fluorescence intensity of each test group detected by the flow cytometer, wherein the fluorescence intensity of the dCas9::Φ group is used as the baseline to calculate the inhibitory effect of other groups. In FIG. 16A, the red fluorescent signal is the autofluorescence of the S. elongatus PCC 7942, which can be used to determine the cellular health status and a relative position of the cell when observing the yellow fluorescence signal. The results show that the yellow fluorescence signal also can be observed in the dCas9 group (expressing dCas9 protein alone) and the P1 group (expressing the sgRNA alone), and the fluorescence intensity of the dCas9 group or the P1 group is similar to the fluorescence intensity of the dCas9::Φ group. However, the yellow fluorescence signal is very weak in the dCas9::P1 group or in the dCas9::NT1 group. It indicates that the gene expression interference system of the S. elongatus PCC 7942 of the present disclosure targeting to the P1 (the position of the promoter) or the NT1 (5′end position of the gene) has an excellent inhibitory effect. Moreover, a slight yellow fluorescence signal can be observed in the dCas9::NT2 group. It indicates that the gene expression interference system of the S. elongatus PCC 7942 of the present disclosure targeting to the NT2 (the position distant from a gene transcription initiation site) has partial inhibitory effect.

In FIG. 16B, there are no significant difference (p>0.05) among the fluorescence intensity of the dCas9 group (263.4 a.u.), the fluorescence intensity of the P1 group (288.7 a.u.) and the fluorescence intensity of the dCas9::Φ group (281.9 a.u.), and no inhibitory effect is observed in these groups. However, the difference between the fluorescence intensity of the groups of the gene expression interference system of the S. elongatus PCC 7942 of the present disclosure and the fluorescence intensity of the dCas9::Q group is statistically significant (p<0.05). Both the dCas9::P1 group (an initiation of the transcription) and the dCas9::NT1 group (an elongation of the transcription) compared to the dCas9::Φ group can effectively inhibit the expression of the target gene (p<0.05), wherein inhibition rates is 95% and 99%, respectively. Besides, the expression of the gene is partially inhibited (p<0.05) in the dCas9::NT2 group (the target site is closer to the middle of the gene), and the inhibition rate is reduced to 76%. The analytical results detected by the flow cytometer are the same as the analytical results observed by the fluorescence microscope. From the above results, the expression of both the dCas9 protein and the sgRNA of the gene expression interference system of the S. elongatus PCC 7942 of the present disclosure are indeed required for the inhibitory effect on the target gene in the S. elongatus PCC 7942 cell. The inhibitory effect can effectively block a binding of a RNA polymerase and the promoter in the initiation of the transcription and interrupt the transcription of the RNA polymerase during the elongation of the transcription to inhibit the expression of the target gene.

### 2.5 Analyses of a Gene Regulation a Stability and a Cytotoxicity of the Gene Expression Interference System of the S. PCC 7942 of the Present Disclosure

To observe whether the gene expression interference system of the S. elongatus PCC 7942 of the present disclosure would cause the cytotoxicity against the S. elongatus PCC 7942 cell, and stably and constitutively inhibit the expression of the gene, the dCas9::4′ group is used as the control to calculate the inhibitory effect of the gene expression interference system of the S. elongatus PCC 7942 of the present disclosure. The tests include the dCas9::P1 group, dCas9::NT1 group and dCas9::NT2 group. The single colony of each test is cultured in 40 ml of the BG-11 medium containing the kanamycin and the chloramphenicol. The wild type S. elongatus PCC 7942 is also cultured in 40 ml of the BG-11 medium as a negative control for observing a growth curve of each group. All groups are cultured 21 days for a long-term observation, and 1 mL of a sample taken from each group is performed a growth curve analysis every day. In addition, 1 mL of the samples taken from each group is analyzed the fluorescent expression level by using flow cytometer analysis every three days, and then another 4 ml of the BG-11 medium is added to maintain a total volume of the medium in the shake flask.

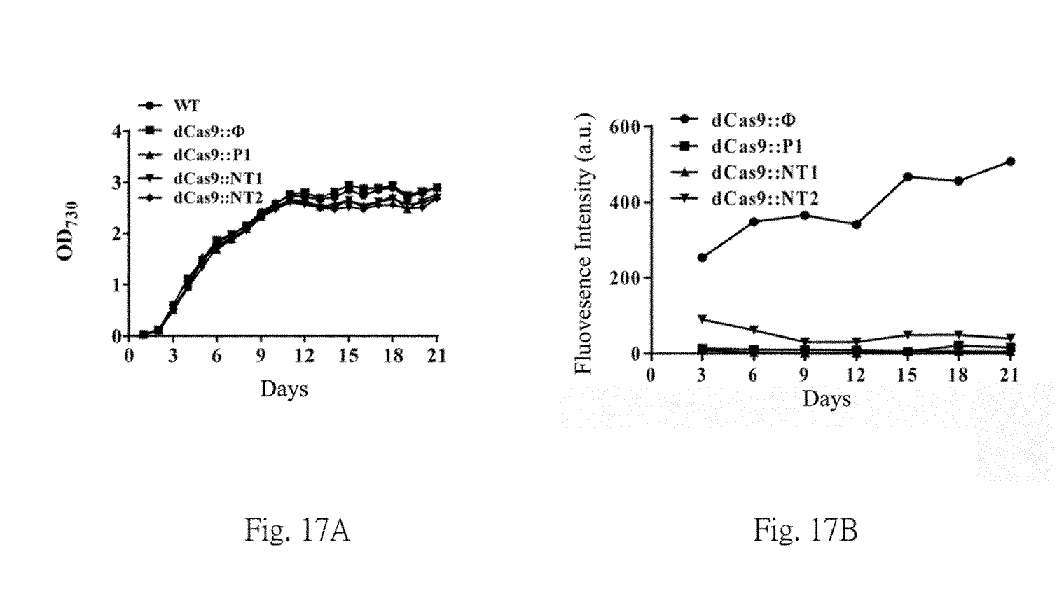

FIG. 17A is analytical result of the cytotoxicity against the S. elongatus PCC 7942 cell affected by the gene expression interference system of the S. elongatus PCC 7942 according to yet another embodiment of the present disclosure. The cytotoxicity against the S. elongatus PCC 7942 cell is observed by the growth curve of the S. elongatus PCC 7942. In FIG. 17A, the growth curve is no significant difference (p>0.05) between all tests and the negative control (the wild type S. elongatus PCC 7942). Thus constitutively expressing the gene expression interference system of the S. elongatus PCC 7942 of the present disclosure does not cause negative effect on the S. elongatus PCC 7942 cell.

FIG. 17B is analytical result of a gene regulation stability of the gene expression interference system of the S. elongatus PCC 7942 according to yet another embodiment of the present disclosure. In FIG. 17B, the gene expression interference system of the S. elongatus PCC 7942 of the present disclosure has the excellent inhibitory effect (96.5%) in an initial growth stage of the of the S. elongatus PCC 7942 (the third day). Even in a late growth stage of the S. elongatus PCC 7942 (the 21^{st} day), the dCas9::NT1 group maintains the excellent inhibitory effect (99%) while the fluorescence expression level of the dCas9::Φ group is continuously increased.

To sum up, the gene expression interference system of the S. elongatus PCC 7942 of the present disclosure and the method for interfering gene expression of the S. elongatus PCC 7942 of the present disclosure not only does not have a negative effect on the S. elongatus PCC 7942 but also can stably and effectively inhibit a long-term expression of the target gene. When compared to conventional methods, the method for interfering gene expression of the S. elongatus PCC 7942 of the present disclosure only need to design the sequence of the sgRNA. The design of the sgRNA is easy, and an inhibition extent of the gene expression can be controlled. Because the gene expression interference system of the S. elongatus PCC 7942 of the present disclosure can inhibit the expression of necessary genes by a partial inhibition of the target gene, it has a potential as a multiplexing. In addition, the CRISPRi system is a gene regulation and editing system of the exogenous gene, it will not compete with an endogenous system of the S. elongatus PCC 7942. Therefore, the gene expression interference system of the S. elongatus PCC 7942 of the present disclosure is expected to become a powerful tool to optimize a protein production in the S. elongatus PCC 7942, which is very advantageous for the protein production in the future.

### III. A Gene Expression Regulation System of the S. elongatus PCC 7942 of the Present Disclosure

### 3.1 Establishment of a CRISPR Gene Expression Regulation System of the S. elongatus PCC 7942

The gene expression regulation system of the S. elongatus PCC 7942 of the present disclosure includes the S. elongatus PCC 7942 cell, a gene editing unit and a gene expression interference unit. The gene editing unit includes a CRISPR/Cas9 expression plasmid and a template plasmid. The gene expression interference unit includes a dCas9 expression plasmid and a sgRNA plasmid.

The CRISPR/Cas9 expression plasmid of the gene editing unit includes a tracrRNA, a Cas9 gene and a crRNA. The template plasmid successively includes a first left homology arm, an first antibiotic resistance gene, an exogenous gene and a first right homology arm, wherein the first left homology arm and the first right homology arm compose a first homology region, a sequence of the first homology region is homologous to a first specific sequence of a chromosome of the S. elongatus PCC 7942, and a sequence of the crRNA is homologous to a second specific sequence of the chromosome of the S. elongatus PCC 7942.

The dCas9 expression plasmid of the gene expression interference unit successively includes a second left homology arm, a first promoter, a dCas9 gene, a second antibiotic resistance gene and a second right homology arm, wherein the second left homology arm and the second right homology arm compose a second homology region. The sgRNA plasmid successively includes a third left homology arm, a second promoter, a sgRNA, a third antibiotic resistance gene and a third right homology arm, wherein the third left homology arm and the third right homology arm compose a third homology region, a sequence of the sgRNA is homologous to a sequence of a target gene, the target gene is on the chromosome of the Synechococcus elongatus PCC 7942 or on an exogenous plasmid, the third homology region and the second homology region are different, and the third antibiotic resistance gene and the 15 second antibiotic resistance gene are different.

The first promoter and the second promoter of the gene expression regulation system of the S. elongatus PCC 7942 of the present disclosure can be Smt promoter, LtetO1 promoter, ConII-ribo promoter, LlacO1 promoter, BAD promoter, Trc promoter, Trc′ promoter, LlacO1′ promoter, ConII promoter, J23101 promoter or J23119 promoter. The first homology region, the second homology region and the third homology region can be neutral site I (NSI) or neutral site II (NSII). The first antibiotic resistance gene, the second antibiotic resistance gene and the third antibiotic resistance gene can be spectinomycin resistance (Spec^{R}) gene, the Km^{R} gene or the Cm^{R} gene.

### 3.2 A Method for Regulating a Gene Expression of the S. elongatus PCC 7942

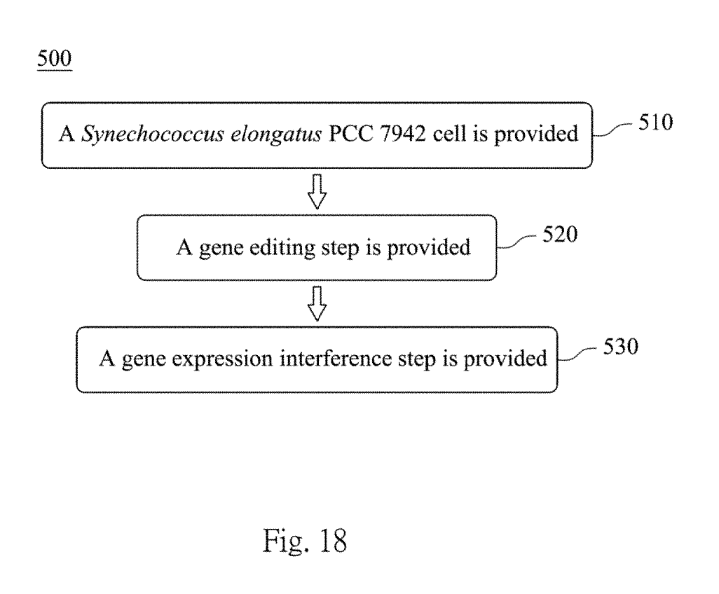

FIG. 18 is a flow diagram showing the method 500 for regulating the gene expression of the S. elongatus PCC 7942 according to yet another embodiment of the present disclosure. In FIG. 18, the method 500 for regulating the gene expression of the S. elongatus PCC 7942 includes a step 510, a step 520 and a step 530.

In the step 510, the S. elongatus PCC 7942 cell is provided.

In the step 520, a gene editing step is provided, wherein the exogenous gene is inserted into the S. elongatus PCC 7942 cell by using the gene editing unit. The gene editing step further includes steps as follows. The CRISPR/Cas9 expression plasmid and the template plasmid are co-transformed into the S. elongatus PCC 7942 cell to obtain a first transformant. The first transformant is cultured, and then the CRISPR/Cas9 expression plasmid therein expresses the tracrRNA, the Cas9 protein and the crRNA to form the Cas9 protein complex, wherein the Cas9 protein complex triggers the double strand break on the second specific sequence of the chromosome of the first transformant, and the first homology region of the template plasmid and the first homology region of the chromosome of the first transformant perform the homologous recombination to insert the first antibiotic resistance gene and the exogenous gene into the first homology region of the chromosome of the first transformant.

In the step 530, a gene expression interference step is provided, wherein an expression of a target gene is inhibited by using the gene expression interference unit. The gene expression interference step further includes steps as follows. The dCas9 expression plasmid is transformed into first transformant to obtain a second transformant, wherein the second homology region of the dCas9 expression plasmid and the second homology region of the chromosome of the second transformant perform the homologous recombination to insert the first promoter, the dCas9 gene and the second antibiotic resistance gene into the second homology region of the chromosome of the second transformant. Then the sgRNA plasmid is transformed into the second transformant to obtain a third transformant, wherein the third homology region of the sgRNA plasmid and the third homology region of the chromosome of the third transformant perform the homologous recombination to insert the second promoter, the sgRNA and the third antibiotic resistance gene into the third homology region of the chromosome of the third transformant. The third transformant is cultured, and an inducer is added to induce the dCas9 expression plasmid therein to express the dCas9 protein, wherein the dCas9 protein and the sgRNA expressed from the sgRNA plasmid form a dCas9 protein complex, and then the dCas9 protein complex bind to the target gene to inhibit the expression of the target gene.

Therefore, the gene expression regulation system of the S. elongatus PCC 7942 of the present disclosure and the method for regulating the gene expression of the S. elongatus PCC 7942 of the present disclosure can comprehensively manipulate the metabolic pathways of the S. elongatus PCC 7942. To optimize the target product yield, the exogenous gene can be inserted into the S. elongatus PCC 7942 cell by using the gene editing unit, and then the expression of the target gene is inhibited by using the gene expression interference unit.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A gene editing system of a Synechococcus elongatus PCC 7942, comprising:
a Synechococcus elongatus PCC 7942 cell;
a CRISPR/Cas9 expression plasmid, which comprises a tracrRNA, a Cas9 gene and a crRNA; and
a template plasmid, which successively comprises a left homology arm, an antibiotic resistance gene, an exogenous gene and a right homology arm;
wherein the left homology arm and the right homology arm compose a homology region, a sequence of the homology region is homologous to a first specific sequence of a chromosome of the Synechococcus elongatus PCC 7942, and a sequence of the crRNA is homologous to a second specific sequence of the chromosome of the Synechococcus elongatus PCC 7942.

2. The gene editing system of the Synechococcus elongatus PCC 7942 of claim 1, wherein the homology region is neutral site I (NSI).

3. The gene editing system of the Synechococcus elongatus PCC 7942 of claim 1, wherein a length of the left homology arm is equal to a length of the right homology arm, which is 400 bp to 700 bp.

4. The gene editing system of the Synechococcus elongatus PCC 7942 of claim 1, wherein the antibiotic resistance gene is spectinomycin resistance (Spec^{R}) gene, kanamycin resistance (Km^{R}) gene or chloramphenicol resistance (Cm^{R}) gene.

5. A gene editing method of a Synechococcus elongatus PCC 7942, comprising:
constructing a CRISPR/Cas9 expression plasmid, which comprises a tracrRNA, a Cas9 gene and a crRNA;
constructing a template plasmid, which successively comprises a left homology arm, an antibiotic resistance gene, an exogenous gene and a right homology arm, wherein the left homology arm and the right homology arm compose a homology region, a sequence of the homology region is homologous to a first specific sequence of a chromosome of the Synechococcus elongatus PCC 7942, and a sequence of the crRNA is homologous to a second specific sequence of the chromosome of the Synechococcus elongatus PCC 7942;
co-transforming the CRISPR/Cas9 expression plasmid and the template plasmid into a Synechococcus elongatus PCC 7942 cell to obtain a transformant; and
culturing the transformant and then the CRISPR/Cas9 expression plasmid therein expressing the tracrRNA, a Cas9 protein and the crRNA to form a Cas9 protein complex, wherein the Cas9 protein complex triggers a double strand break on the second specific sequence of the chromosome of the transformant, and the homology region of the template plasmid and the homology region of the chromosome of the transformant perform a homologous recombination to insert the antibiotic resistance gene and the exogenous gene into the homology region of the chromosome of the transformant.

6. The gene editing method of the Synechococcus elongatus PCC 7942 of claim 5, further comprising a selection step, wherein the transformant is cultured in a medium containing an antibiotic.

7. The gene editing method of the Synechococcus elongatus PCC 7942 of claim 6, wherein the antibiotic is spectinomycin, kanamycin or chloramphenicol.

8. The gene editing method of the Synechococcus elongatus PCC 7942 of claim 5, wherein the homology region is neutral site I (NSI).

9. A gene expression interference system of a Synechococcus elongatus PCC 7942, comprising:
a Synechococcus elongatus PCC 7942 cell;
a dCas9 expression plasmid, which successively comprises a first left homology arm, a first promoter, a dCas9 gene, a first antibiotic resistance gene and a first right homology arm;
wherein the first left homology arm and the first right homology arm compose a first homology region; and
a sgRNA plasmid, which successively comprises a second left homology arm, a second promoter, a sgRNA, a second antibiotic resistance gene and a second right homology arm;
wherein the second left homology arm and the second right homology arm compose a second homology region, a sequence of the sgRNA is homologous to a sequence of a target gene, the target gene is on a chromosome of the Synechococcus elongatus PCC 7942 or on an exogenous plasmid, the second homology region and the first homology region are different, and the second antibiotic resistance gene and the first antibiotic resistance gene are different.

10. The gene expression interference system of the Synechococcus elongatus PCC 7942 of claim 9, wherein the first homology region is neutral site I (NSI) or neutral site II (NSII).

11. The gene expression interference system of the Synechococcus elongatus PCC 7942 of claim 9, wherein the second homology region is neutral site I (NSI) or neutral site II (NSII).

12. The gene expression interference system of the Synechococcus elongatus PCC 7942 of claim 9, wherein the first antibiotic resistance gene is spectinomycin resistance (Spec^{R}) gene, kanamycin resistance (Km^{R}) gene or chloramphenicol resistance (Cm^{R}) gene.

13. The gene expression interference system of the Synechococcus elongatus PCC 7942 of claim 9, wherein the second antibiotic resistance gene is spectinomycin resistance (Spec^{R}) gene, kanamycin resistance (Km^{R}) gene or chloramphenicol resistance (Cm^{R}) gene.

14. The gene expression interference system of the Synechococcus elongatus PCC 7942 of claim 9, wherein the first promoter is Smt promoter, LtetO1 promoter, ConII-ribo promoter, LlacO1 promoter, BAD promoter, Trc promoter, Trc′ promoter, LlacO1′ promoter, ConII promoter, J23101 promoter or J23119 promoter.

15. The gene expression interference system of the Synechococcus elongatus PCC 7942 of claim 9, wherein the second promoter is Smt promoter, LtetO1 promoter, ConII-ribo promoter, LlacO1 promoter, BAD promoter, Trc promoter, Trc′ promoter, LlacO1′ promoter, ConII promoter, J23101 promoter or J23119 promoter.

16. A method for interfering gene expression of a Synechococcus elongatus PCC 7942, comprising:
constructing a dCas9 expression plasmid, which successively comprises a first left homology arm, a first promoter, a dCas9 gene, a first antibiotic resistance gene and a first right homology arm, wherein the first left homology arm and the first right homology arm compose a first homology region;
constructing a sgRNA plasmid, which successively comprises a second left homology arm, a second promoter, a sgRNA, a second antibiotic resistance gene and a second right homology arm, wherein the second left homology arm and the second right homology arm compose a second homology region, a sequence of the sgRNA is homologous to a sequence of a target gene, the target gene is on a chromosome of the Synechococcus elongatus PCC 7942 or on an exogenous plasmid, the second homology region and the first homology region are different, and the second antibiotic resistance gene and the first antibiotic resistance gene are different;
transforming the dCas9 expression plasmid into a Synechococcus elongatus PCC 7942 cell to obtain a first transformant, wherein the first homology region of the dCas9 expression plasmid and the first homology region of the chromosome of the first transformant perform a homologous recombination to insert the first promoter, the dCas9 gene and the first antibiotic resistance gene into the first homology region of the chromosome of the first transformant;
transforming the sgRNA plasmid into the first transformant to obtain a second transformant, wherein the second homology region of the sgRNA plasmid and the second homology region of the chromosome of the second transformant perform the homologous recombination to insert the second promoter, the sgRNA and the second antibiotic resistance gene into the second homology region of the chromosome of the second transformant; and
culturing the second transformant and adding an inducer to induce the dCas9 expression plasmid therein to express a dCas9 protein, wherein the dCas9 protein and the sgRNA expressed from the sgRNA plasmid form a dCas9 protein complex, and then the dCas9 protein complex bind to a target gene to inhibit the expression of the target gene.

17. The method for interfering gene expression of the Synechococcus elongatus PCC 7942 of claim 16, further comprising a first selection step, wherein the first transformant is cultured in a medium containing a first antibiotic.

18. The method for interfering gene expression of the Synechococcus elongatus PCC 7942 of claim 17, wherein the first antibiotic is kanamycin, chloramphenicol or spectinomycin.

19. The method for interfering gene expression of the Synechococcus elongatus PCC 7942 of claim 16, further comprising a second selection step, wherein the second transformant is cultured in the medium containing a second antibiotic.

20. The method for interfering gene expression of the Synechococcus elongatus PCC 7942 of claim 19, wherein the second antibiotic is kanamycin, chloramphenicol or spectinomycin.

21. A gene expression regulation system of a Synechococcus elongatus PCC 7942, comprising:
a Synechococcus elongatus PCC 7942 cell;
a gene editing unit, comprising:
a CRISPR/Cas9 expression plasmid, which comprises a tracrRNA, a Cas9 gene and a crRNA; and
a template plasmid, which successively comprises a first left homology arm, an first antibiotic resistance gene, an exogenous gene and a first right homology arm, wherein the first left homology arm and the first right homology arm compose a first homology region, a sequence of the first homology region is homologous to a first specific sequence of a chromosome of the Synechococcus elongatus PCC 7942, and a sequence of the crRNA is homologous to a second specific sequence of the chromosome of the Synechococcus elongatus PCC 7942; and
a gene expression interference unit, comprising:
a dCas9 expression plasmid, which successively comprises a second left homology arm, a first promoter, a dCas9 gene, a second antibiotic resistance gene and a second right homology arm, wherein the second left homology arm and the second right homology arm compose a second homology region; and
a sgRNA plasmid, which successively comprises a third left homology arm, a second promoter, a sgRNA, a third antibiotic resistance gene and a third right homology arm, wherein the third left homology arm and the third right homology arm compose a third homology region, a sequence of the sgRNA is homologous to a sequence of a target gene, the target gene is on the chromosome of the Synechococcus elongatus PCC 7942 or on an exogenous plasmid, the third homology region and the second homology region are different, and the third antibiotic resistance gene and the second antibiotic resistance gene are different.

22. The gene expression regulation system of the Synechococcus elongatus PCC 7942 of claim 21, wherein the first homology region is neutral site I (NSI) or neutral site II (NSII).

23. The gene expression regulation system of the Synechococcus elongatus PCC 7942 of claim 21, wherein the second homology region is neutral site I (NSI) or neutral site II (NSII).

24. The gene expression regulation system of the Synechococcus elongatus PCC 7942 of claim 21, wherein the third homology region is neutral site I (NSI) or neutral site II (NSII).

25. The gene expression regulation system of the Synechococcus elongatus PCC 7942 of claim 21, wherein the first antibiotic resistance gene is spectinomycin resistance (Spec^{R}) gene, kanamycin resistance (Km^{R}) gene or chloramphenicol resistance (Cm^{R}) gene.

26. The gene expression regulation system of the Synechococcus elongatus PCC 7942 of claim 21, wherein the second antibiotic resistance gene is spectinomycin resistance (Spec^{R}) gene, kanamycin resistance (Km^{R}) gene or chloramphenicol resistance (Cm^{R}) gene.

27. The gene expression regulation system of the Synechococcus elongatus PCC 7942 of claim 21, wherein the third antibiotic resistance gene is spectinomycin resistance (Spec^{R}) gene, kanamycin resistance (Km^{R}) gene or chloramphenicol resistance (Cm^{R}) gene.

28. The gene expression regulation system of the Synechococcus elongatus PCC 7942 of claim 21, wherein the first promoter is Smt promoter, LtetO1 promoter, ConII-ribo promoter, LlacO1 promoter, BAD promoter, Trc promoter, Trc′ promoter, LlacO1′ promoter, ConII promoter, J23101 promoter or J23119 promoter.

29. The gene expression regulation system of the Synechococcus elongatus PCC 7942 of claim 21, wherein the second promoter is Smt promoter, LtetO1 promoter, ConII-ribo promoter, LlacO1 promoter, BAD promoter, Trc promoter, Trc′ promoter, LlacO1′ promoter, ConII promoter, J23101 promoter or J23119 promoter.

30. A method for regulating a gene expression of a Synechococcus elongatus PCC 7942, comprising:
providing the gene expression regulation system of the Synechococcus elongatus PCC 7942 of claim 21;
providing a gene editing step, wherein the exogenous gene is inserted into the Synechococcus elongatus PCC 7942 cell by using the gene editing unit, and the gene editing step comprises:
co-transforming the CRISPR/Cas9 expression plasmid and the template plasmid into the Synechococcus elongatus PCC 7942 cell to obtain a first transformant; and
culturing the first transformant and then the CRISPR/Cas9 expression plasmid therein expressing the tracrRNA, a Cas9 protein and the crRNA to form a Cas9 protein complex, wherein the Cas9 protein complex triggers a double strand break on the second specific sequence of the chromosome of the first transformant, and the first homology region of the template plasmid and the first homology region of the chromosome of the first transformant perform a homologous recombination to insert the first antibiotic resistance gene and the exogenous gene into the first homology region of the chromosome of the first transformant; and
providing a gene expression interference step, wherein an expression of a target gene is inhibited by using the gene expression interference unit, and the gene expression interference step comprises:
transforming the dCas9 expression plasmid into first transformant to obtain a second transformant, wherein the second homology region of the dCas9 expression plasmid and the second homology region of the chromosome of the second transformant perform the homologous recombination to insert the first promoter, the dCas9 gene and the second antibiotic resistance gene into the second homology region of the chromosome of the second transformant;
transforming the sgRNA plasmid into the second transformant to obtain a third transformant, wherein the third homology region of the sgRNA plasmid and the third homology region of the chromosome of the third transformant perform the homologous recombination to insert the second promoter, the sgRNA and the third antibiotic resistance gene into the third homology region of the chromosome of the third transformant; and
culturing the third transformant and adding an inducer to induce the dCas9 expression plasmid therein to express a dCas9 protein, wherein the dCas9 protein and the sgRNA expressed from the sgRNA plasmid form a dCas9 protein complex, and then the dCas9 protein complex bind to the target gene to inhibit the expression of the target gene.

31. The method for regulating gene expression of the Synechococcus elongatus PCC 7942 of claim 30, further comprising a first selection step, wherein the first transformant is cultured in a medium containing a third antibiotic.

32. The method for regulating gene expression of the Synechococcus elongatus PCC 7942 of claim 31, wherein the first antibiotic is kanamycin, chloramphenicol or spectinomycin.

33. The method for regulating gene expression of the Synechococcus elongatus PCC 7942 of claim 30, further comprising a second selection step, wherein the second transformant is cultured in the medium containing a second antibiotic.

34. The method for regulating gene expression of the Synechococcus elongatus PCC 7942 of claim 33, wherein the second antibiotic is kanamycin, chloramphenicol or spectinomycin.

35. The method for regulating gene expression of the Synechococcus elongatus PCC 7942 of claim 30, further comprising a third selection step, wherein the third transformant is cultured in the medium containing a third antibiotic.

36. The method for regulating gene expression of the Synechococcus elongatus PCC 7942 of claim 35, wherein the third antibiotic is kanamycin, chloramphenicol or spectinomycin.
